(19) United States
(12) Patent Application Publication
(10) Pub. No.: US 2019/0328665 A1
(43) Pub. Date: Oct. 31, 2019

(54) LIPOSOME COMPOSITIONS ENCAPSULATING MODIFIED CYCLODEXTRIN COMPLEXES AND USES THEREOF

(71) Applicant: The Johns Hopkins University, Baltimore, MD (US)

(72) Inventors: Bert Vogelstein, Baltimore, MD (US); Kenneth W. Kinzler, Baltimore, MD (US); Shibin Zhou, Baltimore, MD (US); Surojit Sur, Gaithersburg, MD (US)

(21) Appl. No.: 16/288,826

(22) Filed: Feb. 28, 2019

Related U.S. Application Data
(63) Continuation of application No. 15/111,644, filed on Jul. 14, 2016, now abandoned.
(60) Provisional application No. 61/927,233, filed on Jan. 14, 2014.

(51) Int. Cl. A61K 9/127; C08B 37/16; A61K 9/00; A61P 35/00; A61K 47/40; A61K 31/525; A61K 31/166; A61K 47/69

(52) U.S. Cl. CPC A61K 9/1271; C08B 37/0012; A61K 9/0019; A61P 35/00; A61K 47/6951; A61K 9/1278; A61K 31/525; A61K 31/166; A61K 47/40

(57) ABSTRACT

The invention provides liposome compositions comprising liposomes encapsulating cyclodextrins that both bear ionizable functional groups, such as on their solvent-exposed surfaces, and encompass therapeutic agents, as well as uses thereof.

### RELATED APPLICATIONS

This application is a continuation of U.S. application Ser. No. 15/111,644, filed on Jul. 14, 2016, which is a national stage filing under 35 U.S.C. § 371 of PCT Application No. PCT/US15/11342, filed on Jan. 14, 2015, which claims the benefit of priority of U.S. Provisional Application No. 61/927,233, filed on Jan. 14, 2014. The entire contents of these applications are hereby incorporated by reference in their entirety.

### STATEMENT OF RIGHTS

This invention was made with government support under Grants CA 043460, CA 057345, and CA 0062924 awarded by the National Institutes of Health (NIH). The U.S. government has certain rights in the invention. This statement is included solely to comply with 37 C.F.R. § 401.14(a)(f)(4) and should not be taken as an assertion or admission that the application discloses and/or claims only one invention.

### BACKGROUND OF THE INVENTION

There is currently wide interest in the development of nanoparticles for drug delivery (Heidel and Davis (2011) Pharm. Res. 28:187-199; Davis et al. (2010) Nature 464:1067-1070; Choi et al. (2010) Proc. Natl. Acad. Sci. U.S.A. 107:1235-1240; Chertok et al. (2013) Mol. Pharm. 10:3531-3543; Hubbell and Langer (2013) Nat. Mater. 12:963-966; Mura et al. (2013) Nat. Mater. 12:991-1003; and Kanasty et al. (2013) Nat. Mater. 12:967-977). This area of research is particularly relevant to cancer drugs, wherein the therapeutic ratio (dose required for effectiveness to dose causing toxicity) is often low. Nanoparticles carrying drugs can increase this therapeutic ratio over that achieved with the free drug through several mechanisms. In particular, drugs delivered by nanoparticles are thought to selectively enhance the concentration of the drugs in tumors as a result of the enhanced permeability and retention (EPR) effect (Peer et al. (2007) Nat. Nanotech. 2:751-760; Gubernator (2011) Exp. Opin. Drug Deliv. 8:565-580; Huwyler et al. (2008) Int. J. Nanomed. 3:21-29; Maruyama et al. (2011) Adv. Drug Deliv. Rev. 63:161-169; Musacchio and Torchilin (2011) Front. Biosci. 16:1388-1412; Baryshnikov (2012) Vest. Ross. Akad. Med. Nauk. 23-31; Torchilin (2005) Nat. Rev. Drug Disc. 4:145-160; Matsumura and Maeda (1986) Cancer Res. 46:6387-6392; Maeda et al. (2013) Adv. Drug Deliv. Rev. 65:71-79; Fang et al. (2003) Adv. Exp. Med. Biol. 519:29-49; and Fang et al. (2011) Adv. Drug Deliv. Rev. 63:136-151). The enhanced permeability results from a leaky tumor vascular system, whereas the enhanced retention results from the disorganized lymphatic system that is characteristic of malignant tumors.

Much current work in this field is devoted to designing novel materials for nanoparticle generation. This new generation of nanoparticles can carry drugs, particularly those that are insoluble in aqueous medium, that are difficult to incorporate into conventional nanoparticles such as liposomes. However, the older generation of nanoparticles has a major practical advantage in that they have been extensively tested in humans and approved by regulatory agencies such as the Food and Drug Administration in the United States and the European Medicines Agency in Europe. Unfortunately, many drugs cannot be easily or effectively loaded into liposomes, thereby compromising their general use.

In general, liposomal drug loading is achieved by either passive or active methods (Gubernator (2011) Exp. Opin. Drug Deliv. 8:565-580; Kita and Dittrich (2011) Exp. Opin. Drug Deliv. 8:329-342; Schwendener and Schott (2010) Method. Mol. Biol. 605:129-138; Fahr and Liu (2007) Exp. Opin. Drug Deliv. 4:403-416; Chandran et al. (1997) Ind. J. Exp. Biol. 35:801-809). Passive loading involves dissolution of dried lipid films in aqueous solutions containing the drug of interest. This approach can only be used for water-soluble drugs, and the efficiency of loading is often low. In contrast, active loading can be extremely efficient, resulting in high intraliposomal concentrations and minimal wastage of precious chemotherapeutic agents (Gubernator (2011) Exp. Opin. Drug Deliv. 8:565-580; Fenske and Cullis (2008) Liposome Nanomed. 5:25-44; and Barenholz (2003) J. Liposome Res. 13:1-8). In active loading, drug internalization into preformed liposomes is typically driven by a transmembrane pH gradient. The pH outside the liposome allows some of the drug to exist in an unionized form, able to migrate across the lipid bilayer. Once inside the liposome, the drug becomes ionized due to the differing pH and becomes trapped there (FIG. 1A). Many reports have emphasized the dependence of liposome loading on the nature of the transmembrane pH gradient, membrane-water partitioning, internal buffering capacity, aqueous solubility of the drug, lipid composition, and other factors (Abraham et al. (2004) J. Control. Rel. 96:449-461; Haran et al. (1993) Biochim. Biophys. Acta 1151:201-215; Madden et al. (1990) Chem. Phys. Lipids 53:37-46; and Zucker et al. (2009) J. Control. Rel. 139:73-80). As described in a recent model (Zucker et al. (2009) J. Control. Rel. 139:73-80), the aqueous solubility of the drug is one of the requirements for efficient active loading. Another key element for the success of remote loading is the presence of weakly basic functional groups on the small molecule.

Only a small fraction of chemotherapeutic agents possesses the features required for active loading with established techniques. Attempts at active loading of such nonionizable drugs into preformed liposomes result in poor loading efficiencies (FIG. 1B). One potential solution to this problem is the addition of weakly basic functional groups to otherwise unloadable drugs, an addition that would provide the charge necessary to drive these drugs across the pH gradient. However, covalent modification of drugs often alters their biological and chemical properties, and is not desirable in many circumstances.

Accordingly, there is a great need in the art to identify liposomal composition encapsulating therapeutic agents having various chemical properties (e.g., nonionic and/or hydrophobic) at high efficiencies and large concentrations.

### SUMMARY OF THE INVENTION

The present invention is based in part on the discovery that cyclodextrins bearing ionizable functional groups (e.g., weakly basic and/or weakly acidic functional groups on their solvent-exposed surfaces, such as liposome internal phase surfaces) are able to efficiently encapsulate a therapeutic agent (e.g., non-ionizable and/or hydrophobic compositions) at high concentrations and that the functionalized cyclodextrins containing the therapeutic agent can themselves be efficiently remotely loaded into liposomes at high concentrations to generate liposome compositions exhibiting unexpectedly reduced toxicity and enhanced efficacy properties when administered in vivo (see, for example, FIG. 1C). Cyclodextrins are a family of cyclic sugars that are commonly used to solubilize hydrophobic drugs (Albers and Muller (1995) Crit. Rev. Therap. Drug Carrier Syst. 12:311-337; Zhang and Ma (2013) Adv. Drug Delivery Rev. 65:1215-1233; Laza-Knoerr et al. (2010) J. Drug Targ. 18:645-656; Challa et al. (2005) AAPS PharmSci. Tech. 6:E329-E357; Uekama et al. (1998) Chem. Rev. 98:2045-2076; Szejtli (1998) Chem. Rev. 98:1743-1754; Stella and He (2008) Toxicol. Pathol. 36:30-42; Rajewski and Stella (1996) J. Pharm. Sci. 85:1142-1169; Thompson (1997) Crit. Rev. Therap. Drug Carrier Sys. 14:1-104; and Irie and Uekama (1997) J. Pharm. Sci. 86:147-162).

In one aspect, a liposome composition comprising a cyclodextrin, a therapeutic agent, and a liposome, wherein the liposome encapsulates a cyclodextrin having at least one hydroxyl chemical group facing the liposome internal phase replaced with an ionizable chemical group and wherein the cyclodextrin encapsulates the therapeutic agent is provided. In one embodiment, at least one α-D-glucopyranoside unit of the cyclodextrin has at least one hydroxyl chemical group selected from the group consisting of C2, C3, and C6 hydroxyl chemical groups that are replaced with an ionizable chemical group. In another embodiment, at least one α-D-glucopyranoside unit of the cyclodextrin has at least two hydroxyl chemical groups selected from the group consisting of C2, C3, and C6 hydroxyl chemical groups that are replaced with ionizable chemical groups. In still another embodiment, the C2, C3, and C6 hydroxyl chemical groups of at least one α-D-glucopyranoside unit of the cyclodextrin that are replaced with ionizable chemical groups. In yet another embodiment, the at least one α-D-glucopyranoside unit of the cyclodextrin is selected from the group consisting of two, three, four, five, six, seven, eight, and all α-D-glucopyranoside units of the cyclodextrin. In another embodiment, the ionizable chemical group is the same at all replaced positions. In still another embodiment, the ionizable chemical group is a weakly basic functional group (e.g., a group X that has a pKa between 6.5 and 8.5 according to CH3-X) or a weakly acidic functional group (e.g., a group Y that has a pKa between 4.0 and 6.5 according to CH₃—Y). In yet another embodiment, the weakly basic or weakly acidic functional groups are selected from the group consisting of amino, ethylene diamino, dimethyl ethylene diamino, dimethyl anilino, dimethyl naphthylamino, succinyl, carboxyl, sulfonyl, and sulphate functional groups. In another embodiment, the cyclodextrin has a pKₐ₁ of between 4.0 and 8.5. In still another embodiment, the composition is a liquid or solid pharmaceutical formulation. In yet another embodiment, the therapeutic agent is neutrally charged or hydrophobic. In another embodiment, the therapeutic agent is a chemotherapeutic agent. In still another embodiment, the therapeutic agent is a small molecule. In yet another embodiment, the cyclodextrin is selected from the group consisting of β-cyclodextrin, α-cyclodextrin, and γ-cyclodextrin. In another embodiment, the cyclodextrin is β-cyclodextrin, α-cyclodextrin.

In another aspect, a kit comprising a liposome composition described herein, and instructions for use, is provided.

In still another aspect, a method of treating a subject having a cancer comprising administering to the subject a therapeutically effective amount of a liposome composition described herein, is provided. In one embodiment, the therapeutic agent is a chemotherapeutic agent. In another embodiment, the liposome composition is administered by injection subcutaneously or intravenously. In still another embodiment, the subject is a mammal. In yet another embodiment, the mammal is a human.

### BRIEF DESCRIPTION OF FIGURES

FIG. 1 contains three panels FIGS. 1A-1C, showing an embodiment of a schematic representation of active loading of a liposome. FIG. 1A shows remote loading of an ionizable hydrophilic drug using a transmembrane pH gradient results in efficient incorporation. FIG. 1B shows that poorly soluble hydrophobic drug result in meager incorporation into pre-formed liposomes under the conditions shown in FIG. 1A. FIG. 1C shows that encapsulation of a poorly soluble drug into an ionizable cyclodextrin (R═H, ionizable alkyl or aryl groups) enhances its water solubility and permits efficient liposomal loading via a pH gradient.

FIG. 2 contains three panels FIGS. 2A-2C, showing embodiments of synthesized ionizable cyclodextrins. FIG. 2A shows an embodiment of a chemical reaction to form some of the presently disclosed synthesized ionizable cyclodextrins. FIG. 2B shows some embodiments of the presently disclosed synthesized ionizable cyclodextrins bearing ionizable groups at their 6′-position. FIG. 2C depicts the toroidal shape of a cyclodextrin.

FIG. 3, contains four panels FIGS. 3A-3D, showing the active loading of modified β-cyclodextrin using a transmembrane pH gradient. FIG. 3A shows fluorescence of 3-cyclodextrin V in relative fluorescence units (RLU) loaded into liposomes with a pH gradient (citrate liposomes) compared to that of the same compound loaded into liposomes in the absence of a pH gradient (PBS liposomes). FIG. 3B shows dynamic light scattering measurements demonstrating a marginal increase in hydrodynamic radius, but no change in the polydispersity index (PDI) of liposomes remotely loaded with cyclodextrin V. FIGS. 3C-3D show cryoTEM images of dansylated β-cyclodextrin V loaded with a pH gradient (citrate liposomes; FIG. 3C) or without a pH gradient (PBS liposomes; FIG. 3D).

FIG. 4 shows the incorporation of dansylated cyclodextrins into citrate liposomes by analyzing fluorescence in relative fluorescence units (RLU) of dansylated I and cyclodextrin IV in citrate liposomes versus control (PBS) liposomes.

FIG. 5 contains four panels FIGS. 5A-5D, show the remote loading of insoluble hydrophobic dyes into liposomes using modified β-cyclodextrins as seen by fluorescence intensity of remotely loaded coumarin 102 (FIG. 5A), coumarin 314 (FIG. 5B), coumarin 334 (FIG. 5C), and cyclohexyl DNP (FIG. 5D). Insets show photographs of the vials containing the liposomes incubated with the cyclodextrin-encapsulated dye (top) or free dye (bottom).

FIG. 6 shows the ability of various cyclodextrins to transfer coumarin 314 into citrate liposomes. Fluorescence in relative fluorescence units (RLU) of uncomplexed coumarin 314 and coumarin 314 complexed with III (ionizable mono-6-ethylenediamino-6′deoxy-cyclodextrin) and I (unionizable β-cyclodextrin) followed by remote loading into citrate liposomes is shown.

FIG. 7 shows the structure and physical properties of BI-2536 and PD-0325901.

FIG. 8, contains three panels FIGS. 8A-8C, showing the loading and activity of the PLK1 inhibitor, BI-2536L. FIG. 8A shows survival data of animals injected with BI-2536 and CYCL-BI-2536. All treated animals (n=5) succumbed overnight to single iv dose (125 mg/kg) of BI-2536 in its free form, while a single i.v. dose of CYCL-BI-2536 did not elicit any signs of acute toxicity at similar doses (125 mg/kg; n=5) or much higher doses (500 mg/kg; n=5). FIG. 8B shows the results of nude mice (n=4 per arm) bearing HCT 116 xenografts treated with 2 i.v. doses (on days indicated by arrows) of (i) empty liposomes, (ii) free BI-2536 (100 mg/kg), (iii) CYCL-BI-2536 (100 mg/kg), and (iv) CYCL-BI-2536 (400 mg/kg). FIG. 8C shows the results of nude mice bearing HCT 116 xenografts treated with a single i.v. dose of (i) empty liposomes, (ii) BI-2536 (100 mg/kg), or (iii) CYCL-BI-2536 (100 mg/kg). Neutrophils were counted before any drug treatment and every 24 hours thereafter. Means and standard deviations (SD) of the neutrophil counts of five mice in each treatment arm are shown.

FIG. 9 shows the tissue biodistribution of CYCL-coumarin 334 at the 2, 24, and 48 hour time points as histograms from left to right for each tissue, respectively, as indicated. Data are presented as the mean and standard deviation.

FIG. 10, contains two panels FIGS. 10A-10B, showing the loading and activity of the MEK1 inhibitor, PD-0325901. FIG. 10A shows survival curves of animals treated with a single dose of PD-0325901 and CYCL-PD-0325901. Nude mice bearing RKO xenografts were treated with a single dose (200 mg/kg) of PD-0325901 in its free form, a single i.v. dose of CYCL-PD-0325901 at a low dose (200 mg/kg; n=5), or at a higher dose (500 mg/kg; n=5). FIG. 10B shows the results of nude mice bearing RKO xenografts treated with 2 i.v. doses (on days indicated by arrows) of (i) blank liposomes, (ii) free PD-0325901 (150 mg/kg), or (iii) CYCL-PD-325901 (250 mg/kg). Liposomal formulations have been reported as equivalents of free drug. The relative tumor volumes and standard deviation of each experimental arm is shown.

FIG. 11 contains three panels FIGS. 11A-11C, showing the anti-tumor activity of CYCL-BI-2536 (FIG. 11A) and CYCL-PD0325901 (FIGS. 11B and 11C) in a second xenograft model. Liposomal formulations have been reported as equivalents of free drug. The relative tumor volumes and standard deviation of each experimental arm is shown.

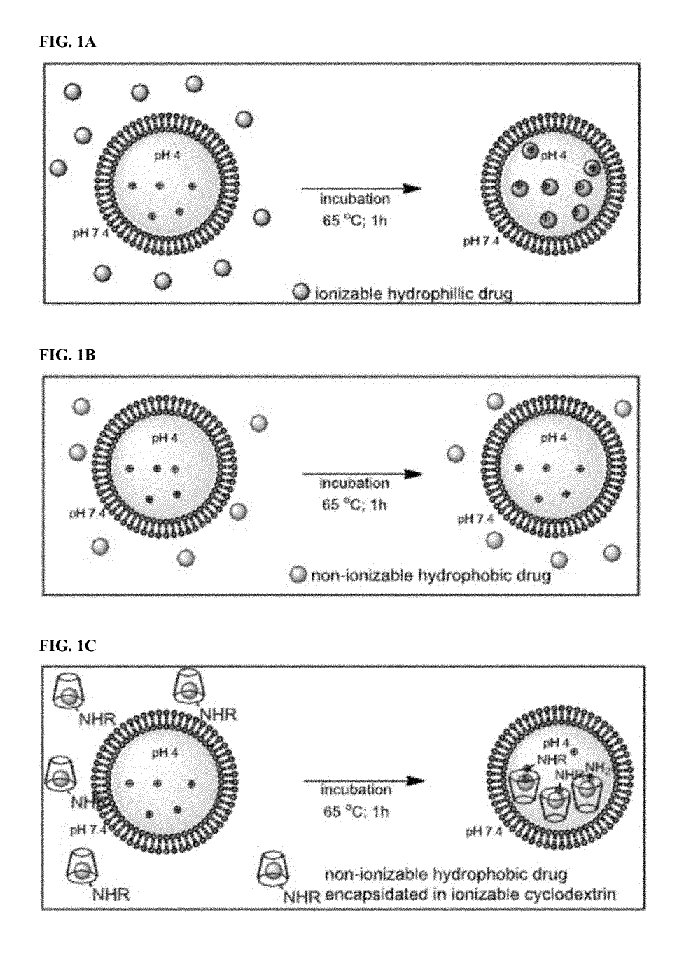

### DETAILED DESCRIPTION OF THE INVENTION

It has been determined herein that cyclodextrins bearing ionizable functional groups (e.g., weakly basic and/or weakly acidic functional groups on their solvent-exposed surfaces, such as liposome internal phase surfaces) are able to efficiently encapsulate a therapeutic agent (e.g., non-ionizable and/or hydrophobic compositions) at high concentrations and that the functionalized cyclodextrins containing the therapeutic agent are efficiently remotely loaded into liposomes at high concentrations to generate liposome compositions exhibiting unexpectedly reduced toxicity and enhanced efficacy properties when administered in vivo. Thus, the present invention provides, at least in part, liposome compositions and kits comprising such modified cyclodextrins and therapeutic agents, as well as methods of making and using such compositions and kits.

### A. Cyclodextrins

The term “cyclodextrin” refers to a family of cyclic oligosaccharides composed of 6 or more α-D-glucopyranoside units linked together by C1-C4 bonds having a toroidal topological structure, wherein the larger and the smaller openings of the toroid expose certain hydroxyl groups of the α-D-glucopyranoside units to the surrounding environment (e.g., solvent). The term “inert cyclodextrin” refers to a cyclodextrin containing α-D-glucopyranoside units having the basic formula C₆H₁₂O₆ and glucose structure without any additional chemical substitutions (e.g., α-cyclodextrin having 6 glucose monomers, β-cyclodextrin having 7 glucose monomers, and γ-cyclodextrin having 8 glucose monomers). The term “cyclodextrin internal phase” refers to the relatively less hydrophilic region enclosed within (i.e., encapsulated by) the toroid topology of the cyclodextrin structure. The term “cyclodextrin external phase” refers to the region not enclosed by the toroid topology of the cyclodextrin structure and can include, for example, the liposome internal phase when the cyclodextrin is encapsulated within a liposome. Cyclodextrins are useful for solubilizing hydrophobic compositions (see, for example, Albers and Muller (1995) Crit. Rev. Therap. Drug Carrier Syst. 12:311-337; Zhang and Ma (2013) Adv. Drug Delivery Rev. 65:1215-1233; Laza-Knoerr et al. (2010) J. Drug Targ. 18:645-656; Challa et al. (2005) AAPS PharmSci. Tech. 6:E329-357; Uekama et al. (1998) Chem. Rev. 98:2045-2076; Szejtli (1998) Chem. Rev. 98:1743-1754; Stella and He (2008) Toxicol. Pathol. 36:30-42; Rajewski and Stella (1996) J. Pharm. Sci. 85:1142-1169; Thompson (1997) Crit. Rev. Therap. Drug Carrier Sys. 14:1-104; and Irie and Uekama (1997) J. Pharm. Sci. 86:147-162). Any substance located within the cyclodextrin internal phase is said to be “encapsulated.”

As used herein, there are no particular limitations on the cyclodextrin so long as the cyclodextrins (a) can encapsulate a desired therapeutic agent and (b) bear ionizable (e.g., weakly basic and/or weakly acidic) functional groups to facilitate encapsulation by liposomes.

For encapsulating a desired therapeutic agent, cyclodextrins can be selected and/or chemically modified according to the characteristics of the desired therapeutic agent and parameters for efficient, high-concentration loading therein. For example, it is preferable that the cyclodextrin itself have high solubility in water in order to facilitate entrapment of a larger amount of the cyclodextrin in the liposome internal phase. In some embodiments, the water solubility of the cyclodextrin is at least 10 mg/mL, 20 mg/mL, 30 mg/mL, 40 mg/mL, 50 mg/mL, 60 mg/mL, 70 mg/mL, 80 mg/mL, 90 mg/mL, 100 mg/mL or higher. Methods for achieving such enhanced water solubility are well known in the art.

In some embodiments, a large association constant with the therapeutic agent is preferable and can be obtained by selecting the number of glucose units in the cyclodextrin based on the size of the therapeutic agent (see, for example, Albers and Muller (1995) Crit. Rev. Therap. Drug Carrier Syst. 12:311-337; Stella and He (2008) Toxicol. Pathol. 36:30-42; and Rajewski and Stella (1996) J. Pharm. Sci. 85:1142-1169). When the association constant depends on pH, the cyclodextrin can be selected such that the association constant becomes large at the pH of the liposome internal phase. As a result, the solubility (nominal solubility) of the therapeutic agent in the presence of cyclodextrin can be further improved. For example, the association constant of the cyclodextrin with the therapeutic agent can be 100, 200, 300, 400, 500, 600, 700, 800, 900, 1,000, or higher.

Derivatives formed by reaction with cyclodextrin hydroxyl groups (e.g., those lining the upper and lower ridges of the toroid of an inert cyclodextrin) are readily prepared and offer a means of modifying the physicochemical properties of the parent (inert) cyclodextrin. It has been determined herein that modifying hydroxyl groups, such as those facing away from the cyclodextrin interior phase, can be replaced with ionizable chemical groups to facilitate loading into liposomes as well as loading of therapeutic agents, such as poorly soluble or hydrophobic agents, within the modified cyclodextrins. In one embodiment, a modified cyclodextrin having at least one hydroxyl group substituted with an ionizable chemical group will result in a charged moiety under certain solvent (e.g., pH) conditions. The term “charged cyclodextrin” refers to a cyclodextrin having one or more of its hydroxyl groups substituted with a charged moiety and the moiety bearing a charge. Such a moiety can itself be a charged group or it can comprise an organic moiety (e.g., a Ci-C₆ alkyl or Ci-C₆ alkyl ether moiety) substituted with one or more charged moieties.

In one embodiment, the “ionizable” or “charged” moieties are weakly ionizable. Weakly ionizable moieties are those that are either weakly basic or weakly acidic. Weakly basic functional groups (X) have a pKa of between about 6.0-9.0, 6.5-8.5, 7.0-8.0, 7.5-8.0, and any range in between inclusive according to CH₃—X. Similarly, weakly acidic functional groups (Y) have a log dissociation constant (pKa) of between about 3.0-7.0, 4.0-6.5, 4.5-6.5, 5.0-6.0, 5.0-5.5, and any range in between inclusive according to CH₃—Y. The pKa parameter is a well-known measurement of acid/base properties of a substance and methods for pKa determination are conventional and routine in the art. For example, the pKa values for many weak acids are tabulated in reference books of chemistry and pharmacology. See, for example, IUPAC Handbook of Pharmaceutical Salts, ed. by P. H. Stahl and C. G Wermuth, Wiley-VCH, 2002; CRC Handbook of Chemistry and Physics, 82nd Edition, ed. by D. R. Lide, CRC Press, Florida, 2001, p. 8-44 to 8-56. Since cyclodextrins with more than one ionizable group have pKa of the second and subsequent groups each denoted with a subscript.

Representative anionic moieties include, without any limitation, carboxylate, carboxymethyl, succinyl, sulfonyl, phosphate, sulfoalkyl ether, sulphate carbonate, thiocarbonate, dithiocarbonate, phosphate, phosphonate, sulfonate, nitrate, and borate groups.

Representative cationic moieties include, without limitation, amino, guanidine, and quarternary ammonium groups.

In another embodiment, the modified cyclodextrin is a “polyanion” or “polycation.” A polyanion is a modified cyclodextrin having more than one negatively charged group resulting in net negative ionic charger of more than two units. A polycation is a modified cyclodextrin having more than one positively charged group resulting in net positive ionic charger of more than two units.

In another embodiment, the modified cyclodextrin is a “chargeable amphiphile.” By “chargeable” is meant that the amphiphile has a pK in the range pH 4 to pH 8 or 8.5. A chargeable amphiphile may therefore be a weak acid or base. By “amphoteric” herein is meant a modified cyclodextrin having a ionizable groups of both anionic and cationic character wherein: 1) at least one, and optionally both, of the cation and anionic amphiphiles is chargeable, having at least one charged group with a pK between 4 and 8 to 8.5, 2) the cationic charge prevails at pH 4, and 3) the anionic charge prevails at pH 8 to 8.5.

In some embodiments, the “ionizable” or “charged” cyclodextrins as a whole, whether polyionic, amphiphilic, or otherwise, are weakly ionizable (i.e., have a pKai of between about 4.0-8.5, 4.5-8.0, 5.0-7.5, 5.5-7.0, 6.0-6.5, and any range in between inclusive).

Any one, some, or all hydroxyl groups of any one, some or all α-D-glucopyranoside units of a cyclodextrin can be modified to an ionizable chemical group as described herein. Since each cyclodextrin hydroxyl group differs in chemical reactivity, reaction with a modifying moiety can produce an amorphous mixture of positional and optical isomers. Alternatively, certain chemistry can allow for pre-modified α-D-glucopyranoside units to be reacted to form uniform products.

The aggregate substitution that occurs is described by a term called the degree of substitution. For example, a 6-ethylenediamino-β-cyclodextrin with a degree of substitution of seven would be composed of a distribution of isomers of 6-ethylenediamino-β-cyclodextrin in which the average number of ethylenediamino groups per 6-ethylenediamino-β-cyclodextrin molecule is seven. Degree of substitution can be determined by mass spectrometry or nuclear magnetic resonance spectroscopy. Theoretically, the maximum degree of substitution is 18 for α-cyclodextrin, 21 for 13, and 24 for γ-cyclodextrin, however, substituents themselves having hydroxyl groups present the possibility for additional hydroxylalkylations. The degree of substitution can be 1, 2, 3, 4, 5, 6, 7, 8, 9, 10, 11, 12, 13, 14, 15, 16, 17, 18, 19, 20, 21, 22, 23, 24, or more and can encompass complete substitution.

Another parameter is the stereochemical location of a given hydroxyl substitution. In one embodiment, at least one hydroxyl facing away from the cyclodextrin interior is substituted with an ionizable chemical group. For example, the C2, C3, C6, C2 and C3, C2 and C6, C3 and C6, and all three of C2-C3-C6 hydroxyls of at least one α-D-glucopyranoside unit are substituted with an ionizable chemical group. Any such combination of hydroxyls can similarly be combined with at least two, three, four, five, six, seven, eight, nine, ten, eleven, up to all of the α-D-glucopyranoside units in the modified cyclodextrin as well as in combination with any degree of substitution described herein.

It is also acceptable to combine one or more of the cyclodextrins described herein.

### B. Liposomes

The term “liposome” refers to a microscopic closed vesicle having an internal phase enclosed by lipid bilayer. A liposome can be a small single-membrane liposome such as a small unilamellar vesicle (SUV), large single-membrane liposome such as a large unilamellar vesicle (LUV), a still larger single-membrane liposome such as a giant unilamellar vesicle (GUV), a multilayer liposome having multiple concentric membranes such as a multilamellar vesicle (MLV), or a liposome having multiple membranes that are irregular and not concentric such as a multivesicular vesicle (MVV). See U.S. Pat. Publ. 2012-0128757; U.S. Pat. Nos. 4,235,871; 4,737,323; WO 96/14057; New (1990) Liposomes: A practical approach, IRL Press, Oxford, pages 33-104; and Lasic (1993) Liposomes from physics to applications, Elsevier Science Publishers BV, Amsterdam for additional description of well-known liposome forms.

The term “liposome internal phase” refers to an aqueous region enclosed within (i.e., encapsulated by) the lipid bilayer of the liposome. By contrast, the term “liposome external phase” refers to the region not enclosed by the lipid bilayer of the liposome, such as the region apart from the internal phase and the lipid bilayer in the case where the liposome is dispersed in liquid.

As used herein, there are no particular limitations on the liposome so long as it can encapsulate the modified cyclodextrins harboring therapeutic agents. In some embodiments, the liposome has a barrier function that prevents the modified cyclodextrin/therapeutic agent complexes from leaking undesirably from the liposome internal phase to the external phase once encapsulated within the liposome internal phase. In the case where it is used as a medicine, it is preferable that the liposome exhibits in vivo stability and has a barrier function that prevents all of the modified cyclodextrin/therapeutic agent complexes from leaking to the liposome external phase in blood when the liposome is administered in vivo.

In some embodiments, the membrane constituents of the liposome include phospholipids and/or phospholipid derivatives. Representative examples of such phospholipids and phospholipid derivatives include, without limitation, phosphatidyl ethanolamine, phosphatidyl choline, phosphatidyl serine, phosphatidyl inositol, phosphatidyl glycerol, cardiolipin, sphingomyelin, ceramide phosphorylethanolamine, ceramide phosphoryl glycerol, ceramide phosphoryl glycerol phosphate, 1,2-dimyristoyl-1,2-deoxyphosphatidyl choline, plasmalogen, and phosphatidic acid. It is also acceptable to combine one or more of these phospholipids and phospholipid derivatives.

There are no particular limitations on fatty-acid residues in the phospholipids and phospholipid derivatives and can include saturated or unsaturated fatty-acid residues having a carbon chain length of 8, 9, 10, 11, 12, 13, 14, 15, 16, 17, 18, 19, 20, or longer. Representative, non-limiting examples include acyl groups derived from fatty-acid such as lauric acid, myristic acid, palmitic acid, stearic acid, oleic acid, and linoleic acid. Phospholipids derived from natural substances such as egg-yolk lecithin and soy lecithin, partially hydrogenated egg-yolk lecithin, (completely) hydrogenated egg-yolk lecithin, partially hydrogenated soy lecithin, and (completely) hydrogenated soy lecithin whose unsaturated fatty-acid residues are partially or completely hydrogenated, and the like, can also be used.

There are no particular limitations on the mixing amount (mole fraction) of the phospholipids and/or phospholipid derivatives that are used when preparing the liposome. In one embodiment, 10 to 80% relative to the entire liposome membrane composition can be used. In another embodiment, a range of between 30 to 60% can be used.

In addition to phospholipids and/or phospholipid derivatives, the liposome can further include sterols, such as cholesterol and cholestanol as membrane stabilizers and fatty acids having saturated or unsaturated acyl groups, such as those having a carbon number of 8, 9, 10, 11, 12, 13, 14, 15, 16, 17, 18, 19, 20, or longer.

There are no particular limitations on the mixing amount (mole fraction) of these sterols that are used when preparing the liposome, but 1 to 60% relative to the entire liposome membrane composition is preferable, 10 to 50% is more preferable, and 30 to 50% is even more preferable. Similarly, there are no particular limitations on the mixing amount (mole fraction) of the fatty acids, but 0 to 30% relative to the entire liposome membrane composition is preferable, 0 to 20% is more preferable, and 0 to 10% is even more preferable. With respect to the mixing amount (mole fraction) of the antioxidants, it is sufficient if an amount is added that can obtain the antioxidant effect, but 0 to 15% of the entire liposome membrane composition is preferable, 0 to 10% is more preferable, and 0 to 5% is even more preferable.

The liposome can also contain functional lipids and modified lipids as membrane constituents. Representative, non-limiting examples of functional lipids include lipid derivatives retained in blood (e.g., glycophorin, ganglioside GM1, ganglioside GM3, glucuronic acid derivatives, glutaminic acid derivatives, polyglycerin phospholipid derivatives, polyethylene glycol derivatives (methoxypolyethylene glycol condensates, etc.) such as N-[carbonyl-methoxy polyethylene glycol-2000]-1,2-dipalmitoyl-sn-glycero-3-phosphoethanolamine, N-[carbonyl-methoxy polyethylene glycol-5000]-1,2-dipalmitoyl-sn-glycero-3-phosphoethanolamine, N-[carbonyl-methoxy polyethylene glycol-750]-1,2-distearoyl-sn glycero-3-phosphoethanolamine, N-[carbonyl-methoxy polyethylene glycol-2000]-1,2-distearoyl-sn-glycero-3-phosphoethanolamine (MPEG 2000-distearoyl phosphatidyl ethanolamine), and N-[carbonyl-methoxy polyethylene glycol-5000]-1,2-distearoyl-sn-glycero-3-phosphoethanolamine, which are condensates of phosphoethanolamine and methoxy polyethylene glycol), temperature-sensitive lipid derivatives (e.g., dipalmitoyl phosphatidylcholine), pH-sensitive lipid derivatives (e.g., dioleoyl phosphatidyl ethanolamine), and the like. Liposomes containing lipid derivatives retained in blood are useful for improving the blood retention of the liposome, because the liposome becomes difficult to capture in the liver as a foreign impurity. Similarly, liposomes containing temperature-sensitive lipid derivatives are useful for causing destruction of liposome at specific temperatures and/or causing changes in the surface properties of the liposome. Furthermore, by combining this with an increase in temperature at the target site, it is possible to destroy the liposome at the target site, and release the therapeutic agent at the target site. Liposomes containing pH-sensitive lipid derivatives are useful for enhancing membrane fusion of liposome and endosome when the liposome is incorporated into cells due to the endocytosis to thereby improve transmission of the therapeutic agent to the cytoplasm.

Representative, non-limiting examples of modified lipids include PEG lipids, sugar lipids, antibody-modified lipids, peptide-modified lipids, and the like. Liposomes containing such modified lipids can be targeted to desired target cells or target tissue. Also, there are no particular limitations on the mixing amount (mole fraction) of functional lipids and modified lipids used when preparing the liposome. In some embodiments, such lipids make up 0-50%, 0-40%, 0-30%, 0-20%, 0-15%, 0-10%, 0-5%, 0-1% or less of the entirety of liposome membrane constituent lipids.

Based on the description above and well-known methods in the art, the composition of the liposome membrane constituents having such membrane permeability at a level allowing practical application can be appropriately selected by those skilled in the art according to the therapeutic agent, target tissue, and the like.

When used as a medicine, it is preferable that the therapeutic agent/cyclodextrin complex be released from the liposome after the liposome reaches the target tissue, cells, or intracellular organelles. It is believed that the liposome compositions described herein contain membrane constituents themselves are ordinarily biodegradable, and ultimately decompose in target tissue or the like and that the encapsulated therapeutic agent/cyclodextrin complex is thereby released through dilution, chemical equilibrium, and/or enzymatic cyclodextrin degradation effects.

Depending on the desired application, the particle size of the liposome can be regulated. For example, when it is intended to transmit liposome to cancerous tissue or inflamed tissue by the Enhanced Permeability and Retention (EPR) effect as an injection product or the like, it is preferable that liposome particle size be 30-400 nm, 50-200 nm, 75-150 nm, and any range in between. In the case where the intention is to transmit liposome to macrophage, it is preferable that liposome particle size be 30 to 1000 nm, and it is more preferable that the particle size be 100 to 400 nm. In the case where liposome composition is to be used as an oral preparation or transdermal preparation, the particle size of liposome can be set at several microns. It should be noted that in normal tissue, vascular walls serve as barriers (because the vascular walls are densely constituted by vascular endothelial cells), and microparticles such as supermolecules and liposome of specified size cannot be distributed within the tissue. However, in diseased tissue, vascular walls are loose (because interstices exist between vascular endothelial cells), increasing vascular permeability, and supermolecules and microparticles can be distributed to extravascular tissue (enhanced permeability). Moreover, the lymphatic system is well developed in normal tissue, but it is known that the lymphatic system is not developed in diseased tissue, and that supermolecules or microparticles, once incorporated, are not recycled through the general system, and are retained in the diseased tissue (enhanced retention), which forms the basis of the EPR effect (Wang et al. (2012) Annu. Rev. Med. 63:185-198; Peer et al. (2007) Nat. Nanotech. 2:751-760; Gubernator (2011) Exp. Opin. Drug Deliv. 8:565-580; Huwyler et al. (2008) Int. J. Nanomed. 3:21-29; Maruyama et al. (2011)Adv. Drug Deliv. Rev. 63:161-169; Musacchio and Torchilin (2011) Front. Biosci. 16:1388-1412; Baryshnikov (2012) Vest. Ross. Akad. Med. Nauk. 23-31; and Torchilin (2005) Nat. Rev. Drug Disc. 4:145-160). Thus, it is possible to control liposome pharmacokinetics by adjusting liposome particle size.

The term “liposome particle size” refers to the weight-average particle size according to a dynamic light scattering method (e.g., quasi-elastic light scattering method).

For example, liposome particle sizes can be measured using dynamic light scattering instruments (e.g., Zetasizer Nano ZS model manufactured by Malvem Instruments Ltd. and ELS-8000 manufactured by Otsuka Electronics Co., Ltd.). The instruments measure Brownian motion of the particles and particle size is determined based on established dynamic light scattering methodological theory.

In addition, there are no particular limitations on the solvent of the liposome internal phase. Exemplary buffer solutions include, without limitation, as phosphate buffer solution, citrate buffer solution, and phosphate-buffered physiological saline solution, physiological saline water, culture mediums for cell culturing, and the like. In the case where buffer solution is used as solvent, it is preferable that the concentration of buffer agent be 5 to 300 mM, 10 to 100 mM, or any range in between. There are also no particular limitations on the pH of the liposome internal phase. In some embodiments, the liposome internal phase has a pH between 2 and 11, 3 and 9, 4 and 7, 4 and 5, and any range in between inclusive.

### C. Therapeutic Agents

There are no particular limitations on the therapeutic agent in the present invention as long as the therapeutic agent is encapsulated by the modified cyclodextrin. For example, it is known that α-cyclodextrin has an internal phase pore diameter size of 0.45-0.6, β-cyclodextrin has an internal phase pore diameter size of 0.6 to 0.8 nm, and γ-cyclodextrin has an internal phase pore diameter size of 0.8 to 0.95 nm. The cyclodextrin can be chosen to match the size of the therapeutic agent to allow for encapsulation. As described above, modifications to the non-carbon cyclodextrin groups (e.g., hydroxyl groups) can be selected to modulate intermolecular interactions between the cyclodextrin and the therapeutic agent to thereby modulate encapsulation of the therapeutic agent by the cyclodextrin.

As therapeutic agents, any desired agent can be used, such as those useful in the fields of medicines (including diagnostic drugs), cosmetic products, food products, and the like. For example, the therapeutic agent can be selected from a variety of known classes of useful agents, including, for example, proteins, peptides, nucleotides, anti-obesity drugs, nutriceuticals, corticosteroids, elastase inhibitors, analgesics, anti-fungals, oncology therapies, anti-emetics, analgesics, cardiovascular agents, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators and xanthines. With respect to therapeutic agents, it is acceptable to combine one or more agents.

In one embodiment, the therapeutic agents can be low-molecular compounds, such as small molecules. Among these, compounds used as antitumor agents, antibacterial agents, anti-inflammatory agents, anti-myocardial infarction agents, and contrast agents are suitable.

With respect to the molecular weight of the therapeutic agent, a range of 100 to 2,000 daltons is preferable, a range of 200 to 1,500 daltons is more preferable, and a range of 300 to 1,000 daltons is even more preferable. Within these ranges, the liposome membrane permeability of the therapeutic agent is generally satisfactory according to the compositions described herein.

There are no particular limitations on anti-neoplastic or anti-tumor agents in the present invention. Representative examples include, without limitation, BI-2536, PD-0325901, camptothecin; taxane; iphosphamide, nimstine hydrochloride, carvocon, cyclophosphamide, dacarbazine, thiotepa, busulfan, melfaran, ranimustine, estramustine phosphate sodium, 6-mercaptopurine riboside, enocitabine, gemcitabine hydrochloride, carmfur, cytarabine, cytarabine ocfosfate, tegafur, doxifluridine, hydroxycarbamide, fluorouracil, methotrexate, mercaptopurine, fludarabine phosphate, actinomycin D, aclarubicin hydrochloride, idarubicin hydrochloride, pirarubicin hydrochloride, epirubicin hydrochloride, daunorubicin hydrochloride, doxorubicin hydrochloride, epirubicin, pirarubicin, daunorubicin, doxorubicin, pirarubicin hydrochloride, bleomycin hydrochloride, zinostatin stimalamer, neocarzinostatin, mitomycin C, bleomycin sulfate, peplomycin sulfate, etoposide, vinorelbine tartrate, vincrestine sulfate, vindesine sulfate, vinblastine sulfate, amrubicin hydrochloride, gefinitib, exemestane, capecitabine, eribulin, eribulin mesylate, and the like. With respect to the compounds recorded as salts among the aforementioned agents, any salt is acceptable and free bodies are also acceptable. With respect to compounds recorded as free bodies, any salt is acceptable.

Similarly, there are no particular limitations on antibacterial agents. Representative examples include, without limitation, amfotericine B, cefotiam hexyl, cephalosporin, chloramphenicol, diclofenac, and the like. With respect to compounds of the aforementioned antibacterial agents, any salt is acceptable.

Also, there are no particular limitations on anti-inflammatory agents. Representative examples include, without limitation, prostaglandins (PGE1 and PGE2), dexamethasone, hydrocortisone, pyroxicam, indomethacin, prednisolone, and the like. With respect to compounds of the aforementioned anti-inflammatory agents, any salt is acceptable.

There are also no particular limitations on anti-myocardial infarction agents. Representative examples include, without limitation, adenosine, atenolol, pilsicamide, and the like. With respect to compounds of the aforementioned anti-myocardial infarction agents, any salt is acceptable.

There are also no particular limitations on contrast agents. Representative examples include, without limitation, iopamidol, ioxaglic acid, iohexyl, iomeprol, and the like. With respect to the contrast agents, any salt is acceptable.

In some embodiments, the therapeutic agent is “poorly water soluble” or “hydrophobic,” which terms are used interchangeably to encompass therapeutic agents that are sparingly soluble in water, as evidenced by a room temperature water solubility of less than about 10 mg/mL, 9 mg/mL, 8 mg/mL, 7 mg/mL, 6 mg/mL, 5 mg/mL, 4 mg/mL, 3 mg/mL, 2 mg/mL, 1 mg/mL, 900 μg/mL, 800 μg/mL, 700 μg/mL, 600 μg/mL, 500 μg/mL, 400 μg/mL, 300 μg/mL, 200 μg/mL, 100 μg/mL, 95 μg/mL, 90 μg/mL, 85 μg/mL, 80 μg/mL, 75 μg/mL, 70 μg/mL, 65 μg/mL, 60 μg/mL, 55 μg/mL, and in some cases less than about 50 μg/mL, or any range in between inclusive. In one embodiment, the term “slightly soluble” is applicable when one part of an agent can be solubilized by 100 to 1000 parts of solvent (e.g., water). It will be appreciated that the room temperature water solubility for any given compound can be easily determined using readily available chemistry techniques and tools, such as high performance liquid chromatography or spectrophotometry.

### D. Liposome Composition

The term “liposome composition” refers to a composition that contains a liposome and that further contains cyclodextrin chemically modified from its inert form and a therapeutic agent in the liposome internal phase. Liposome compositions can include solid and liquid forms. In the case where the liposome composition is in a solid form, it can be made into a liquid form by dissolving or suspending it in a prescribed solvent. In the case where the liposome composition is frozen solid, it can be made into a liquid form by melting by leaving it standing at room temperature.

The concentration of liposome and the concentration of the therapeutic agent in the liposome composition can be appropriately set according to the liposome composition objective, formulation, and other considerations well known to the skilled artisan. In the case where the liposome composition is a liquid formulation, the concentration of liposome as the concentration of all lipids constituting the liposome may be set at 0.2 to 100 mM, and preferably at 1 to 30 mM. The concentration (dosage) of therapeutic agent in the case where the liposome composition is used as a medicine is described below. With respect to the quantity of cyclodextrin in the liposome composition, it is preferable that it be 0.1 to 1000 mol equivalent relative to the therapeutic agent, and it is more preferable that it be 1 to 100 mol equivalent relative to the therapeutic agent.

There are no particular limitations on the solvent of the liposome composition in the case where the liposome composition is a liquid formulation. Representative examples include, without limitation, buffer solutions such as phosphate buffer solution, citrate buffer solution, and phosphate-buffered physiological saline solution, physiological saline water, and culture mediums for cell culturing. There are also no particular limitations on the pH of the liposome external phase of the liposome composition. In some embodiments, such as pH is between 2 and 11, 3 and 10, 4 and 9, 7.4, 7.0, or any pH higher than that of the liposome internal phase.

In some embodiments, pharmaceutically excipients can be added, such as sugar, such as monosaccharides such as glucose, galactose, mannose, fructose, inositol, ribose, and xylose; disaccharides such as lactose, sucrose, cellobiose, trehalose, and maltose; trisaccharides such as raffinose and melezitose; polysaccharides such as cyclodextrin; and sugar alcohols such as erythritol, xylitol, sortibol, mannitol and maltitol; polyvalent alcohols such as glycerin, diglycerin, polyglycerin, propylene glycol, polypropylene glycol, ethylene glycol, diethylene glycol, triethylene glycol, polyethylene glycol, ethylene glycol monoalkylether, diethylene glycol monoalkylether, 1,3-butylene glycol. Combinations of sugar and alcohol can also be used.

For purposes of stable long-term storage of the liposome that is dispersed in solvent, from the standpoint of physical stability including coagulation and so on, it is preferable to eliminate the electrolyte in the solvent as much as possible. Moreover, from the standpoint of chemical stability of the lipids, it is preferable to set the pH of the solvent from acidic to the vicinity of neutral (pH 3.0 to 8.0), and to remove dissolved oxygen through nitrogen bubbling. Representative examples of liquid stabilizers include, without limitation, normal saline, isotonic dextrose, isotonic sucrose, Ringer's solution, and Hanks' solution. A buffer substance can be added to provide pH optimal for storage stability. For example, pH between about 6.0 and about 7.5, more preferably pH about 6.5, is optimal for the stability of liposome membrane lipids, and provides for excellent retention of the entrapped entities.

Histidine, hydroxyethylpiperazine-ethylsulfonate (HEPES), morpholipo-ethylsulfonate (MES), succinate, tartrate, and citrate, typically at 2-20 mM concentration, are exemplary buffer substances. Other suitable carriers include, e.g., water, buffered aqueous solution, 0.4% NaCl, 0.3% glycine, and the like. Protein, carbohydrate, or polymeric stabilizers and tonicity adjusters can be added, e.g., gelatin, albumin, dextran, or polyvinylpyrrolidone. The tonicity of the composition can be adjusted to the physiological level of 0.25-0.35 mol/kg with glucose or a more inert compound such as lactose, sucrose, mannitol, or dextrin. These compositions can be sterilized by conventional, well known sterilization techniques, e.g., by filtration. The resulting aqueous solutions can be packaged for use or filtered under aseptic conditions and lyophilized, the lyophilized preparation being combined with a sterile aqueous medium prior to administration.

There are no particular limitations on the concentration of the sugar contained in the liposome composition, but in a state where the liposome is dispersed in a solvent, for example, it is preferable that the concentration of sugar be 2 to 20% (W/V), and 5 to 10% (W/V) is more preferable. With respect to the concentration of polyvalent alcohol, 1 to 5% (W/V) is preferable, and 2 to 2.5% (W/V) is more preferable.

Solid formulations of liposome compositions can also include pharmaceutical excipients. Such components can include, for example, sugar, such as monosaccharides such as glucose, galactose, mannose, fructose, inositole, ribose, and xylose; disaccharides such as lactose, sucrose, cellobiose, trehalose, and maltose; trisaccharides such as raffinose and melezitose; polysaccharides such as cyclodextrin; and sugar alcohols such as erythritol, xylitol, sorbitol, mannitol, and maltitol. More preferable are blends of glucose, lactose, sucrose, trehalose, and sorbitol. Even more preferable are blends of lactose, sucrose, and trehalose. By this refers to, solid formulations can be stably stored over long periods. When frozen, it is preferable that solid formulations contain polyvalent alcohols (aqueous solutions) such as glycerin, diglycerin, polyglycerin, propylene glycol, polypropylene glycol, ethylene glycol, diethylene glycol, triethylene glycol, polyethylene glycol, ethylene glycol monoalkylether, diethylene glycol monoalkylether and 1,3-butylene glycol. With respect to polyvalent alcohols (aqueous solutions), glycerin, propylene glycol, and polyethylene glycol are preferable, and glycerin and propylene glycol are more preferable. By this refers to, it is possible to stably store the solid formulation over long periods. Sugars and polyvalent alcohols can be used in combination.

The liposome compositions described herein can further be characterized according to entity-to-lipid ratio. In general, the entity-to-lipid ratio, e.g., therapeutic agent load ratio obtained upon loading an agent depends on the amount of the agent entrapped inside the liposomes, the concentration of ions in active loading processes, and the physicochemical properties of the ions and the type of counter-ion used. Because of high loading efficiencies achieved in the compositions and/or by the methods of the present invention, the entity-to-lipid ratio for the entity entrapped in the liposomes is over 70%, 75%, 80%, 85%, 90%, 95%, 99%, or more calculated on the basis of the amount of the entity and the liposome lipid taken into the loading process (the “input” ratio). It is also possible to achieve 100% (quantitative) encapsulation.

The entity-to lipid ratio in the liposomes can be characterized in terms of weight ratio (weight amount of the entity per weight or molar unit of the liposome lipid) or molar ratio (moles of the entity per weight or molar unit of the liposome lipid). One unit of the entity-to-lipid ratio can be converted to other units by a routine calculation, as exemplified below. The weight ratio of an entity in the liposome compositions described herein is typically at least 0.05, 0.1, 0.2, 0.35, 0.5, or at least 0.65 mg of the entity per mg of lipid. In terms of molar ratio, the entity-to-lipid ratio according to the present invention is at least from about 0.02, to about 5, preferably at least 0.1 to about 2, and more preferably, from about 0.15 to about 1.5 moles of the drug per mole of the liposome lipid.

In one embodiment, the entity-to-lipid ratio is at least 0.1 mole of therapeutic agent per mole of liposome lipid, and preferably at least 0.2, 0.3, 0.4, 0.5, or more.

The liposome compositions of the present invention can further be characterized by their unexpected combination of high efficiency of the entrapped therapeutic agent and low toxicity. In general, the activity of a therapeutic agent liposomally encapsulated according to the present invention, e.g., the anti-neoplastic activity of an anti-cancer therapeutic agent in a mammal in a mammal, is at least equal to, at least 2, 2.5, 3, 3.5, 4, 4.5, 5 or more times higher, or at least such fold higher than the activity of the therapeutic entity if it is administered in the same amount via its routine non-liposome formulation, e.g., without using the liposome composition of the present invention, while the toxicity of the liposomally encapsulated entity does not exceed, is at least twice, at least three times, or at least four times lower than that of the same therapeutic entity administered in the same dose and schedule but in a free, non-encapsulated form.

### E. Methods of Making Liposome Compositions

Numerous methods are well known in the art for preparing liposomes. Representative examples include, without limitation, the lipid film method (Vortex method), reverse phase evaporation method, ultrasonic method, pre-vesicle method, ethanol injection method, French press method, cholic acid removal method, Triton X-100 batch method, Ca²⁺ fusion method, ether injection method, annealing method, freeze-thaw method, and the like.

The various conditions (quantities of membrane constituents, temperature, etc.) in liposome preparation can be suitably selected according to the liposome preparation method, target liposome composition, particle size, etc. However, cyclodextrin is known to have the effect of removing lipid (particularly, cholesterol, etc.) from liposomes. It is therefore preferable that the amount of lipid used in the liposome preparation be set in consideration of this effect.

The therapeutic agent/cyclodextrin complexes can be obtained by agitating and mixing the cyclodextrin (e.g., a solution containing the cyclodextrin) upon dropwise addition of the therapeutic agent (e.g., a solution containing the therapeutic agent) or vice versa. It is possible to use a substance dissolved in a solvent or a solid substance as the therapeutic agent according to the physical properties of the therapeutic agent. There are no particular limitations on the solvent, and one can use, for example, a substance identical to the liposome external phase. The amount of the therapeutic agent that is mixed with the cyclodextrin can be equimolar quantities or in different ratios depending on the desired level of incorporation. In some embodiments, absolute amounts of therapeutic agent can range between 0.001 to 10 mol equivalents, 0.01 to 1 mol equivalent, or any range inclusive relative to the amount of cyclodextrin. Also, there are no particular limitations on the heating temperature. For example, 5° C. or higher, room temperature or higher (e.g., 20° C. or higher is also preferable) or the phase transition temperature of the lipid bilayer membrane of the liposome or higher, are all acceptable.

The liposome particle size can be optionally adjusted as necessary. Particle size can be adjusted, for example, by conducting extrusion (extrusion filtration) under high pressure using a membrane filter of regular pore diameter. Particle size adjustment can be conducted at any timing during manufacture of the liposome composition. For example, particle size adjustment can be conducted before introducing the therapeutic agent/cyclodextrin complexes into the liposome internal phase or after the therapeutic agent/cyclodextrin complexes have been remotely loaded into the liposome internal phase.

Well-known methods exist for removing any undesired or unincorporated complexes or compositions, such as therapeutic agent not encapsulated by cyclodextrins or therapeutic agent cyclodextrin complexes not encapsulated by liposomes. Representative examples include, without limitation, dialysis, centrifugal separation, and gel filtration.

Dialysis can be conducted, for example, using a dialysis membrane. As a dialysis membrane, one may cite a membrane with molecular weight cut-off such as a cellulose tube or Spectra/Por. With respect to centrifugal separation, centrifugal acceleration any be conducted preferably at 100,000 g or higher, and more preferably at 300,000 g or higher. Gel filtration may be carried out, for example, by conducting fractionation based on molecular weight using a column such as Sephadex or Sepharose.

In some embodiments, an active remote loading method can be used to encapsulate therapeutic agent/cyclodextrin complexes within a liposome. Generally, the presence of an ionic gradient (e.g., titratable ammonium, such as unsubstituted ammonium ion) in the inner space of a liposome can provide enhanced encapsulation of weak amphiphilic bases, for example, via a mechanism of “active”, “remote”, or “transmembrane gradient-driven” loading (Haran, et al., Biochim. Biophys. Acta, 1993, v. 1152, p. 253-258; Maurer-Spurej, et al., Biochim. Biophys. Acta, 1999, v. 1416, p. 1-10).

For example, active remote loading can be achieved by using a transmembrane pH gradient. The liposome internal and external phases differ in pH by 1-5 pH units, 2-4 pH units, 0.5 pH unit, 1 pH unit, 2 pH units, 3 pH units, 3.4 pH units, 4 pH units, 5 pH units, 6 pH units, 7 pH units, or any range inclusive. Either the liposome internal or external phase can have the higher pH according to the type of the therapeutic agent and the ionizable groups on the modified cyclodextrins. On the other hand, it is also acceptable if the liposome internal and external phases do not substantially have difference in pH (i.e., the liposome external and internal phases have substantially the same pH).

The pH gradient can be adjusted by using a compound conventionally known in the art used in pH gradient methods. Representative examples include, without limitation, amino acids such as arginine, histidine, and glycine; acids such as ascorbic acid, benzoic acid, citric acid, glutamic acid, phosphoric acid, acetic acid, propionic acid, tartaric acid, carbonic acid, lactic acid, boric acid, maleic acid, fumaric acid, malic acid, adipic acid, hydrochloric acid, and sulfuric acid; salts of the aforementioned acids such as sodium salt, potassium salt, and ammonium salt; and alkaline compounds such as tris-hydroxymethylamino methane, ammonia water, sodium hydride, potassium hydride, and the like.

Many different ions that can be used in the ion gradient method. Representative example include, without limitation, ammonium sulfate, ammonium chloride, ammonium borate, ammonium formate, ammonium acetate, ammonium citrate, ammonium tartrate, ammonium succinate, ammonium phosphate, and the like. Moreover, with respect to the ion gradient method, the ion concentration of the liposome internal phase can be selected appropriately according to the type of the therapeutic agent. A higher ion concentration is more preferable and is preferably 10 mM or higher, more preferably 20 mM or higher, even more preferably 50 mM or higher. Either the liposome internal or external phase can have the higher ion concentration according to the type of the therapeutic agent. On the other hand, it is also acceptable if the liposome internal and external phases do not substantially have difference in ion concentration, i.e., the liposome external and internal phases have substantially the same ion concentration. The ion gradient can also be adjusted by substituting or diluting the liposome external phase.

In one embodiment, a step in which the membrane permeability of the liposome is enhanced can be added using well-known methods. Representative examples include, without limitation, heating liposome-containing compositions, adding a membrane fluidizer to liposome-containing compositions, and the like.

In the case where liposome-containing compositions, such as a solution, are heated, the therapeutic agent/cyclodextrin complexes can generally be more efficiently introduced into the liposome internal phase by heating to higher temperatures. Specifically, it is preferable to set the temperature of heating taking into consideration the thermal stability of the therapeutic agent/cyclodextrin complexes and the employed liposome membrane constituents. In particular, it is preferable that the temperature of heating be set to the phase transition temperature of the lipid bilayer membrane of the liposome or higher.

The term “phase transition temperature” of the lipid bilayer membrane of liposome refers to the temperature at which heat absorption starts (the temperature when endothermic reaction begins) in differential thermal analysis of elevated temperatures conditions. Differential thermal analysis is a technique enabling analysis of the thermal properties of specimens by measuring the temperature difference between a specimen and reference substance as a function of time or temperature while changing the temperature of the specimen and reference substance. In the case where differential thermal analysis is conducted with respect to liposome membrane constituents, the liposome membrane components fluidize as temperature increases, and endothermic reaction is observed. The temperature range in which endothermic reaction is observed greatly varies according to the liposome membrane components. For example, in the case where liposome membrane components consist of a pure lipid, the temperature range in which endothermic reaction is observed is extremely narrow, and endothermic reaction is often observed within a range of ±1° C. relative to the endothermic peak temperature. On the other hand, in the case where liposome membrane components consist of multiple lipids, and particularly in the case where liposome membrane components consist of lipids derived from natural materials, the temperature range in which endothermic reaction is observed tends to widen, and endothermic reaction is observed, for example, within a range of ±5° C. relative to the endothermic peak temperature (that is, a broad peak is observed). As liposome membrane fluidization is increased, membrane permeability of the therapeutic agent/cyclodextrin complexes is increased by raising the temperature higher than the phase transition temperature of the liposome lipid bilayer membrane.

For example, although dependent on the thermal stability of the therapeutic agent/cyclodextrin complexes and the employed liposome membrane constituents, the temperature ranges in some embodiments can be from the phase transition temperature of the liposome lipid bilayer membrane to +20° C., +10° C., +5° C., or less, or any range in between such as +5° C. to +10° C. of such a phase transition temperature. In general, the heating temperature can ordinarily range between 20 to 100° C., 40 to 80° C., 45 to 65° C., and any range in between. It is preferable that the heating temperature is higher than or equal to the phase transition temperature.

In the heating step, there are no particular limitations on the time during which the temperature is maintained at or above the phase transition temperature, and this may be properly set within a range, for example, of several seconds to 30 minutes. Taking into consideration the thermal stability of the therapeutic agent and lipids as well as efficient mass production, it is desirable to conduct the treatment within a short time. That is, it is preferable that the elevated temperature maintenance period be 1 to 30 minutes, and 2 minutes to 5 minutes is more preferable. However, these temperature maintenance times in no way limit the present invention.

Moreover, as stated above, it is also possible to enhance liposome membrane permeability by adding a membrane fluidizer to the obtained mixed solution (that is, adding it to the external phase side of the liposome). Representative examples include, without limitation, organic solvents, surfactants, enzymes, etc. that are soluble in aqueous solvents. Representative organic solvents include, without limitation, monovalent alcohols such as ethyl alcohol and benzyl alcohol; polyvalent alcohols such as glycerin and propylene glycol; aprotic polar solvents such as dimethyl sulfoxide (DMSO). Representative surfactants include, without limitation, anionic surfactants such as fatty acid sodium, monoalkyl sulfate, and monoalkyl phosphate; cationic surfactants such as alkyl trimethyl ammonium salt; ampholytic surfactants such as alkyl dimethylamine oxide; and non-ionic surfactants such as polyoxyethylene alkylether, alkyl monoglyceryl ether, and fatty acid sorbitan ester. Representative enzymes include, without limitation, cholinesterase and cholesterol oxidase. Those skilled in the art can set the quantity of membrane fluidizer according to the composition of liposome membrane constituents, the membrane fluidizer, and the like, taking into consideration the degree of efficiency of entrapment of the therapeutic agent due to addition of the membrane fluidizer, the stability of the liposome, etc.

Methods of making liposome compositions described herein can further include a step of adjusting the liposome external phase of the obtained liposome composition and/or a step of drying the obtained liposome composition before and/or after encapsulation of the therapeutic agent/cyclodextrin complexes.

For example, the liposome external phase in the liquid liposome composition can be adjusted (replaced, etc.) to make a final liposome composition if it is to be used as a liquid formulation. Where the liposome composition is to be made into a solid preparation, the liquid liposome composition obtained in the above-mentioned introduction step can be dried to make the final solid liposome composition. Freeze drying and spray drying are representative, non-limiting examples of methods for drying the liposome composition. In cases where the liposome composition is a solid preparation, it can be dissolved or suspended in a suitable solvent and used as a liquid formulation. The solvent for use can be appropriately set according to the purpose of use for the liposome composition. For example, in the case of using the liposome composition as an injection product, the solvent can be sterile distilled water or other solvent compatible with injection. In the case of using the liposome composition as a medicine, the physician or patient can inject the solvent into a vial into which the solid preparation is entrapped, for example, to make the preparation at the time of use. In the case where the liquid liposome composition is a frozen solid preparation, it can be stored in a frozen state, and put in use as a liquid formulation by returning it to a liquid state by leaving it to melt at room temperature or by rapidly melting it with heat at the time of use.

### F. Pharmaceutical Compositions and Methods of Administration

The liposome compositions described herein can be used as a pharmaceutical composition such as a therapeutic composition or a diagnostic composition in the medical field. For example, the liposome composition can be used as a therapeutic composition by incorporating an antineoplastic agent as the therapeutic agent and can be used as a diagnostic composition by incorporating contrast agent as the therapeutic agent. The liposome composition can also be used for any number of other purposes, such as a cosmetic product or as a food additive.

Typically, the liposome pharmaceutical composition of the present invention is prepared as a topical or an injectable, either as a liquid solution or suspension. However, solid forms suitable for solution in, or suspension in, liquid vehicles prior to injection can also be prepared. The composition can also be formulated into an enteric-coated tablet or gel capsule according to known methods in the art.

In the case where the liposome composition of the present invention is used as a pharmaceutical composition, the liposome composition can be administered by injection (intravenous, intra-arterial, or local injection), orally, nasally, subcutaneously, pulmonarily, or through eye drops, and in particular local injection to a targeted group of cells or organ or other such injection is preferable in addition to intravenous injection, subcutaneous injection, intracutaneous injection, and intra-arterial injection. Tablet, powder, granulation, syrup, capsule, liquid, and the like may be given as examples of the formulation of the liposome composition in the case of oral administration. Injection product, drip infusion, eye drop, ointment, suppository, suspension, cataplasm, lotion, aerosol, plaster, and the like can be given as examples of formulations of the liposome composition in the case of non-oral administration, and an injection product and drip infusion agent are particularly preferable.

When the liposome composition is used as a cosmetic product, as the form of the cosmetic product, one may cite, for example, lotions, creams, toners, moisturizers, foams, foundations, lipsticks, face packs, skin washes, shampoos, rinses, conditioners, hair tonics, hair liquids, hair creams, etc.

The term “administering” a substance, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to an animal by any suitable route for delivery of the therapeutic agent to the desired location in the animal, including in any way which is medically acceptable which may depend on the condition or injury being treated. Possible administration routes include injections, by parenteral routes such as intramuscular, subcutaneous, intravenous, intraarterial, intraperitoneal, intraarticular, intraepidural, intrathecal, or others, as well as oral, nasal, ophthalmic, rectal, vaginal, topical, or pulmonary, e.g., by inhalation. For the delivery of liposomally drugs formulated according to the invention, to tumors of the central nervous system, a slow, sustained intracranial infusion of the liposomes directly into the tumor (a convection-enhanced delivery, or CED) is of particular advantage (Saito et al. (2004) Cancer Res. 64:2572-2579; Mamot et al. (2004) J. Neuro-Oncology 68:1-9). The compositions can also be directly applied to tissue surfaces. Sustained release, pH dependent release, or other specific chemical or environmental condition mediated release administration is also specifically included in the invention, e.g., by such means as depot injections, or erodible implants.

The dosage of the pharmaceutical composition upon administration can differ depending on the type of target disease, the type of the therapeutic agent, as well as the age, sex, and weight of the patient, the severity of the symptoms, along with other factors. It is to be understood that the determination of the appropriate dose regimen for any given therapeutic agent encapsulated within the liposomes and for a given patient is well within the skill of the attending physician. For example, the quantity of liposome pharmaceutical composition necessary to deliver a therapeutically effective dose can be determined by routine in vitro and in vivo methods, common in the art of drug testing (e.g., D. B. Budman, A. H. Calvert, E. K. Rowinsky (editors). Handbook of Anticancer Drug Development, L W W, 2003).

Alternatively, the attending physician can rely on the recommended dose for the given drug when administered in free form. Generally, therapeutically effective dosages for various therapeutic entities are well known to those of skill in the art. Typically the dosages for the liposome pharmaceutical composition of the present invention range between about 0.005 and about 500 mg of the therapeutic entity per kilogram of body weight, most often, between about 0.1 and about 100 mg therapeutic entity/kg of body weight.

### G. Kit

According to the present invention, a kit is provided for preparing the liposome composition. The kit can be used to prepare the liposome composition as a therapeutic or diagnostic, which can be used by a physician or technician in a clinical setting or a patient. The kit includes a liposome reagent. The liposome reagent can be either in a solid or a liquid form. If the liposome reagent is in a solid form, the liposome reagent can be dissolved or suspended in an appropriate solvent to obtain the liposome, and the above-mentioned liposome dispersion liquid can be dried to obtain the liposome reagent. Drying can be carried out similarly to the above-mentioned drying of the liposome composition. When using the kit, if the liposome reagent is in a solid form, the liposome regent can be dissolved or suspended in an appropriate solvent to make the liposome dispersion liquid. When doing so, the solvent is similar to the liposome external phase in the above-mentioned liposome dispersion liquid.

The kit of the present invention preferably further contains a therapeutic agent. The therapeutic agent can be either in a solid or liquid form (a state of dissolved or suspended in a solvent). When using the kit, if the therapeutic agent is in a solid form, it is preferable that it be dissolved or suspended in an appropriate solvent to make a liquid form. The solvent can be appropriately set according to the physical properties and the like of the therapeutic agent, and may be made similar to the liposome external phase in the above-mentioned liposome dispersion liquid, for example.

In the kit, the liposome reagent and the therapeutic agent can be packaged separately, or they may be in solid forms and mixed together.

In the case where the liposome reagent and the therapeutic agent are both in solid forms and are packaged together, the mixture of the liposome reagent and the therapeutic agent is appropriately dissolved or suspended in a solvent. When doing so, the solvent is similar to the liposome external phase in the above-mentioned liposome dispersion liquid. It is thereby possible to form a state in which the liposome dispersion liquid and the therapeutic agent are mixed, after which use is made possible by carrying out other steps in the introduction of the therapeutic agent in the liposome internal phase of the liposome dispersion liquid in the manufacturing method of the above-mentioned liposome composition.

In another embodiment, the kit can comprise a liposome composition described herein including directions for use.

### Exemplification

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The following Examples are offered by way of illustration and not by way of limitation.

### Example 1: Materials and Methods for Examples 2-4

A. General method for synthesis of ionizable β-cyclodextrins

β-Cyclodextrin (Sigma-Aldrich, St. Louis, Mo.) was monotosylated with 0.9 molar equivalent of tosyl chloride in pyridine at the primary 6′ hydroxyl group to afford the corresponding tosylate, which was converted to the iodo-derivative by treatment with sodium iodide in acetone. The iodo derivative was converted to the desired aminated cyclodextrin by heating at 80° C. for 8-12 h with the appropriate amine (Tang and Ng (2008) Nat. Protocol. 3:691-697). 6′-mono-succinyl-β-cyclodextrin was synthesized by treatment of parent β-cyclodextrin with 0.9 equivalents of succinic anhydride in DMF (Cucinotta et al. (2005) J. Pharmaceut. Biomed. Anal. 37:1009-1014). The product was precipitated in acetone and purified by HPLC before use. 6′,6′,6′,6′,6′,6′,6′-heptakis-succinyl-β-cyclodextrin was synthesized from β-cyclodextrin by treatment with excess succinic anhydride in DMF and precipitated with acetone. Fractional crystallization afforded the desired compound in ˜85% purity.

Dansylated cyclodextrins I, IV, and V were synthesized from commercially available 1-cyclodextrin and compounds II and III, respectively, by treatment with a 0.9 molar equivalent of dansyl chloride in pyridine.

Each intermediate and the final product was purified by HPLC using a preparative C18 column and linear gradients of 0-95% solvent B (acetonitrile) in solvent A (water). All cyclodextrins were characterized by ¹H NMR and electrospray ionization (ESI) MS and matched with previously published literature references.

6′,6′,6′,6′,6′,6′,6′-heptakis-amino-β-cyclodextrin was purchased from CTD holdings and used without further purification.

BI-2536 (Hoffmann et al. (2004) WO Published Patent Appl. 2004-076454) and PD-0325901 (Warmus et al. (2008) Bioorga. Med. Chem. Lett. 18:6171-617) were synthesized as previously described.

B. General Procedure for the Preparation of Liposomes

Hydrogenated egg phosphatidylcholine (Avanti Polar Lipids), cholesterol and 1,2-distearoyl-sn-glycero-3-phosphoethanolamine-N-[methoxy(polyethylene glycol)-2000 (DSPE-PEG 2000) (Avanti Polar Lipids) (molar ratios, 50:45:5) were dissolved in chloroform (20 ml). The solvent was removed in vacuo to give a thin lipid film, which was hydrated by shaking in the appropriate buffer (PBS, pH 7.4; 200 mM citrate, pH4.0; or 80 mM Arg-HEPES, pH 9.0) at 50° C. for 2 hours. The vesicle suspension was sonicated for 30 minutes and then extruded successively through 0.4-, 0.2-, and 0.1-μm polycarbonate membranes (Whatman, Nucleopore Track-Etch Membrane) at 50° C. to obtain the final liposomes. The transmembrane gradient was then created by equilibrium dialysis of the liposomes against 300 mM sucrose or phosphate-buffered saline (PBS) overnight. The average size and polydispersity index was then measure by Dynamic Light Scattering (DLS) on a Zetasizer Nano ZS90 (Malvern Instruments) at a wavelength of 633 nm and a 90° detection angle.

C. Protocol for Passive Loading of Liposomes

Method 1: Encapsulation of BI-2536 in the lipid layer. Hydrogenated egg phosphatidylcholine (Avanti Polar Lipids), cholesterol, and DSPE-PEG2000 (Avanti Polar Lipids) (molar ratios 50:45:5) were dissolved in chloroform (20 mL). Ten milligrams of BI-2536 (in 1 mL chloroform) was added and the solvent was evaporated to generate a thin film. One milliliter of PBS (pH 7.4) was added to hydrate the lipid layer, and the mixture was shaken at 50° C. for 2 hours as described above. The vesicle suspension was sonicated for 30 min and then extruded successively through 0.4-, 0.2-, and 0.1-μm polycarbonate membranes (Whatman; Nuclepore Track-Etched Membrane) at 50° C. to obtain the final liposomes with low polydispersity at the desired size. The liposomes were then dialyzed in PBS overnight to remove unentrapped drug. The average size and polydispersity index were then measured by dynamic light-scattering experiments on a Malvem Z90. Drug content was calculated by rupturing the liposomes with an equal volume of methanol and measuring the UV-vis absorbance on a NanoDrop 1000.

Method 2: Hydration of the lipid layer with an aqueous formulation of BI-2536. Hydrogenated egg phosphatidylcholine (Avanti Polar Lipids), cholesterol, and DSPE-PEG2000 (Avanti Polar Lipids) (molar ratios 50:45:5) were dissolved in chloroform (20 mL), and the solvent was evaporated in vacuo to generate a thin film. One milliliter of aqueous BI-2536 (4 mg; pH 5.5) was added to hydrate the lipid layer, and the mixture was shaken at 50° C. for 2 hours as described above. The vesicle suspension was sonicated for 30 minutes and then extruded successively through 0.4-, 0.2-, and 0.1-μm polycarbonate membranes (Whatman; Nuclepore Track-Etched Membrane) at 50° C. to obtain the final liposomes with low polydispersity at the desired size. The liposomes were then dialyzed against PBS overnight to remove unentrapped drug. The average size and polydispersity index were then measured by dynamic light-scattering experiments on a Malvem Z90. Drug content was calculated by rupturing the liposomes with an equal volume of methanol and measuring the UV-vis absorbance on a NanoDrop 1000.

D. General Procedure of Preparation of Encapsulated Complexes

Equimolar quantities of the drug (0.1 mmol; 30-50 mg) and appropriate cyclodextrin (0.11 mmol; 110-185 mg) were dissolved separately in methanol (nearly saturated; ˜1-2 mL) and deionized water (˜10-20 mg/mL), respectively. The methanolic solution of the drug was then added dropwise into the cyclodextrin solution with agitation ensuring a uniform suspension. This suspension was then shaken at 55° C. for 36-48 hours using an Eppendorf Thermomixer R. The solution was filtered to remove particulate matter and flash frozen in a dry ice/acetone bath followed by lyophilization. The lyophilized complex was stored at −20° C. until further use.

E. General Protocol for Remote Loading of Liposomes

The lyophilized powder complex described above was pulverized and incubated with appropriate liposomal solutions (30-40 mg drug equivalent in 6 mL liposomal solution, to achieve loading ratios of 5-8 mg/mL concentrations) for 1 hour at 65° C. They were centrifuged at 1,000×g for 3 minutes to remove particulate matter and then dialyzed against 300 mM sucrose or commercial PBS solution (pH 7.4) overnight to remove material that had not been loaded into the liposomes. The size distributions of the liposomal formulations were characterized using the Malvem ZS90 instrument described above. Concentrations of BI-2536 and PD-0325901 in liposomes were measured in triplicate using a Nanodrop 100 after disruption of the liposomal solutions with equal volumes of methanol at 367 nm for BI-2536 and 277 nm for PD-0325901.

F. Tissue Biodistribution Studies

Coumarin 334 was used as a drug surrogate to assess biodistribution and pharmacokinetics of cyclodextrin-encapsulated liposomes. Coumarin 334 (3 mg) was dissolved in methanol (6 mL) and added dropwise to an aqueous solution of cyclodextrin VI (14 mg in 20 mL water). The solution was shaken at 55° C. for 48 hours and lyophilized. The lyophilized powder was incubated with citrate liposomes (internal pH 4.0) at 65° C. for 1 hour. The liposomal solution was dialyzed against PBS overnight. To assess loading efficiency, 100 μL liposomes was broken with 100 μL methanol and analyzed for fluorescence. The loading efficiency was found to be 90%. Female athymic nu/nu mice bearing HCT116 subcutaneous xenografts were used in the study following a modified protocol described in Macdiarmid et al. (2007) Cancer Cell 11:431-445. When the tumor volumes reached 400-600 mm³, 12 mice were treated intravenouslys (i.v.) with 200 μL cyclodextrin-encapsulated, liposomal (CYCL-)coumarin 334 (0.5 mg/mL). Posttreatment, four mice were euthanized at time points 2, 24, and 48 hours and tumor, spleen, liver, kidneys, heart, and lungs were excised and weighed. Blood was also collected, and plasma was separated and stored at 4° C. Except for plasma, each frozen tissue was homogenized and sonicated in 0.9% saline [3×volume (μL) of tissue mass (mg)]. Methanol was added to a final volume of 33% (vol/vol) with vortexing. The samples were centrifuged (6,000×g for 10 minutes) and the fluorescence in the supematants was measured by a CytoFluor II fluorescence multiwall plate reader (Applied Biosystems) using excitation 485 nm/emission 530 nm. As a control for tissue autofluorescence, tumor-bearing animals treated with equivalent volumes of empty liposomes were euthanized and their tissue and plasma were harvested.

G. In Vivo Mouse Treatment

Five million HCT116 (p53^{-/-}), HCT116 (p53+/+), or RKO cells were injected subcutaneously (s.c.) into the flanks of female athymic nu/nu mice and allowed to grow for three weeks, reaching 300-400 mm³ in volume. In the case of CYCL-BI-2536, the animals were then randomly segregated into four arms. In all cases, liposomal formulations (CYCL-drug) have been reported as equivalents of free drug. Over the course of 2 weeks, the first arm received empty liposomes; the second arm received a single dose of 100 mg/kg formulation of the free drug twice using a formulation reported in the literature (at day 0 and day 7); the third and fourth arms received 100 mg/kg and 400 mg/kg, respectively, of the CYCL-BI-2536 liposomal formulation at the same time points. Tumor volume was recorded every 48 hours. The average tumor size for each respective group was normalized to the tumor volume at the first day of treatment. In case of CYCL-PD-0325901, the first arm was treated twice with empty liposomes at day 0 and day 8, whereas the other two arms received two doses of free PD-0325901 and CYCL-PD-0325901, respectively, at the same time points. For clarity of the experimental outcome, the data are presented as the average tumor size of each group normalized to the tumor volume on day 0. The tumor regression experiments in each case were terminated and the animals euthanized when the tumors on the control animals reached 2,000 mm³.

### Example 2: Remote Loading of Chemically-Modified 3-Cyclodextrins into Liposomes

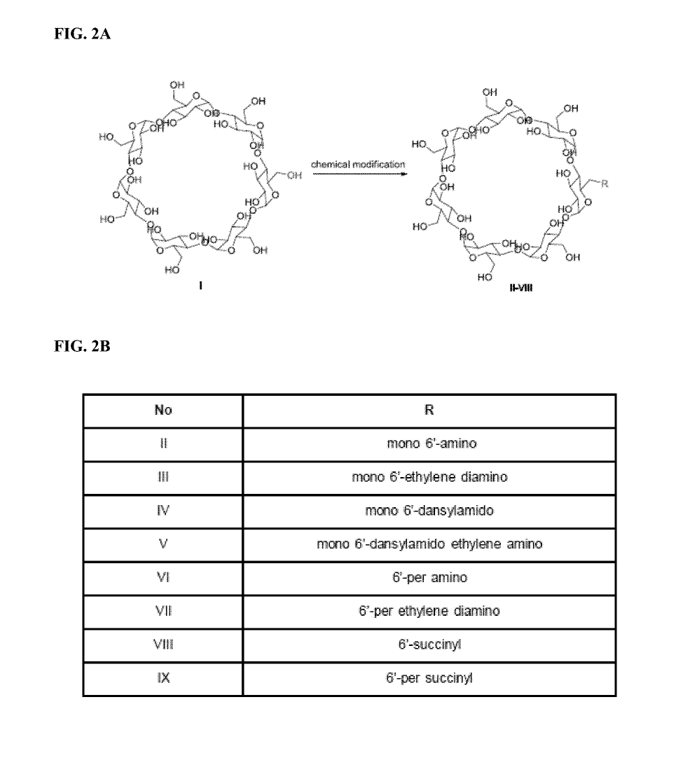

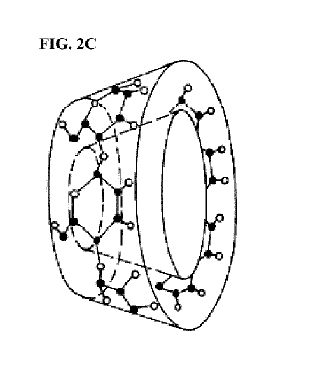

A set of modified β-cyclodextrins bearing ionizable groups at their 6′-position were designed and synthesized (FIG. 2). In analogs II-V, the 6′ primary hydroxyl moiety was modified to an amino group, an ethylenediamino group, or a fluorescent version of either, whereas analog VIII involved introduction of a succinyl group in that position. The rest of the analogs (VI, VII and IX) had all seven primary hydroxyls replaced by amino, ethylene-diamino, or succinyl moieties. All analogs were purified by HPLC and characterized by MS and NMR spectra. Appropriate negative controls were synthesized by introducing similarly sized chemical modifications not containing ionizable groups.

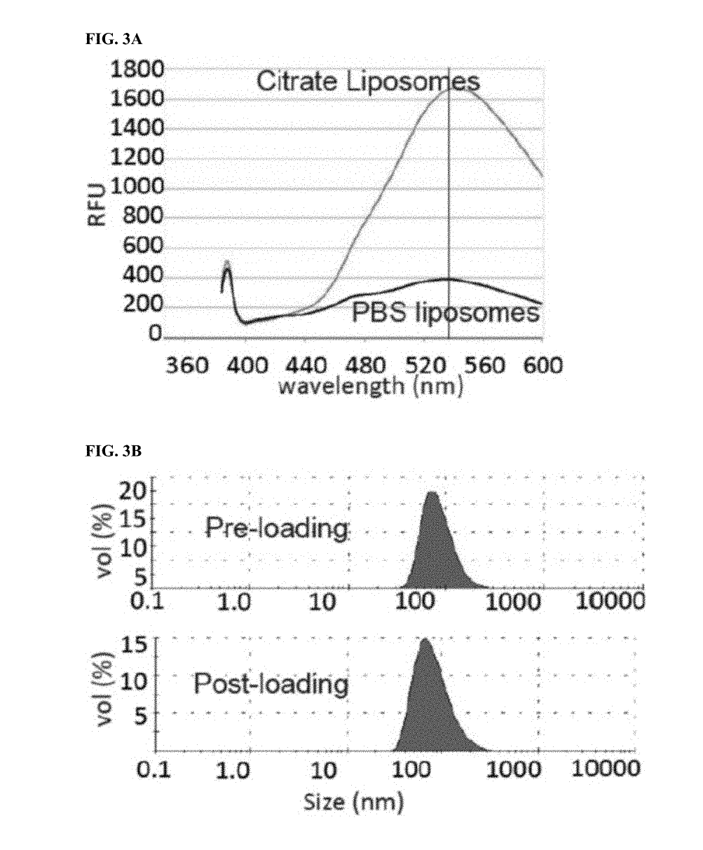

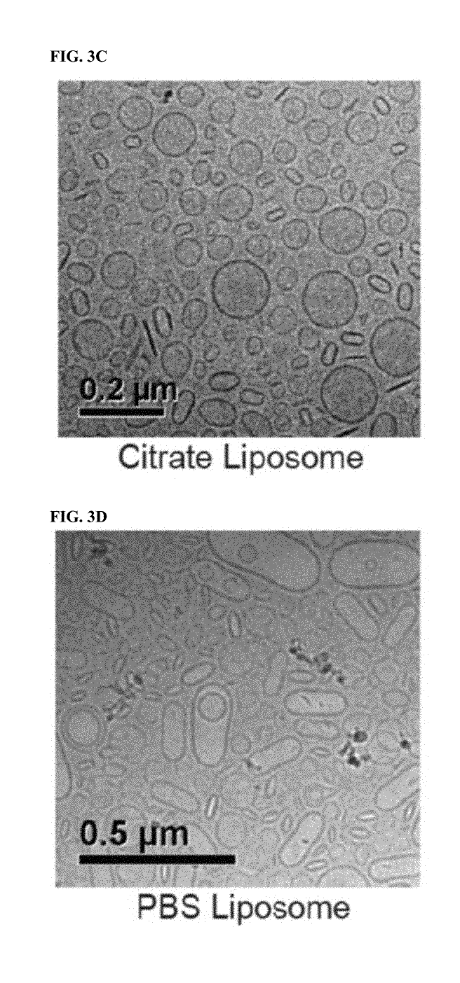

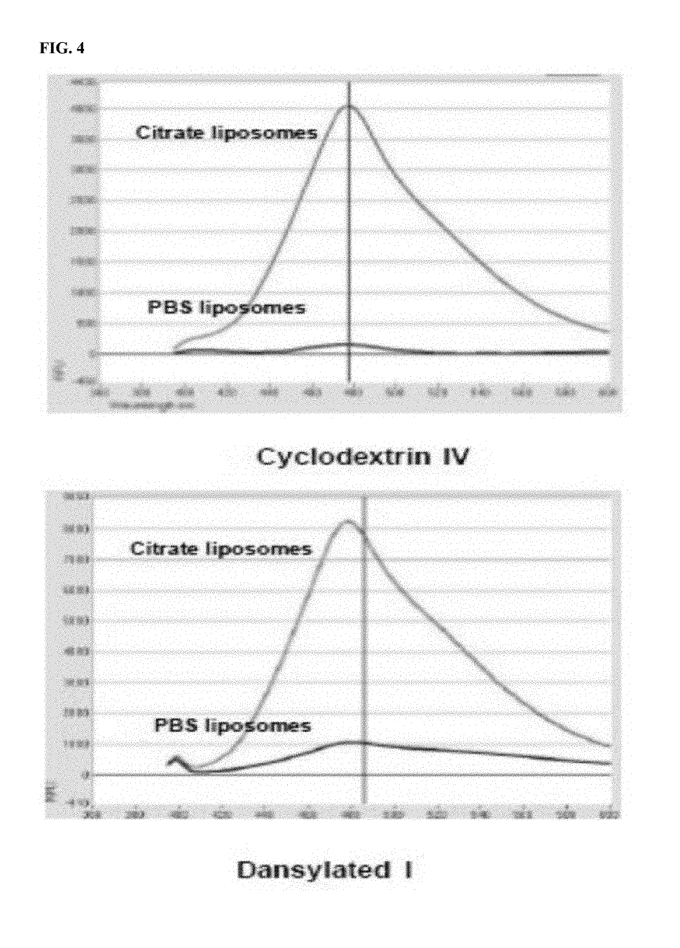

Two fluorescent (dansylated) cyclodextrins (compounds IV and V) were tested for their ability to be loaded into liposomes. The liposomes were generated by hydrating lipid films with 200 mM citrate buffer, so that their internal pH was 4.0. These liposomes were then dialyzed in PBS (pH 7.4) to remove the citrate from outside the liposomes and were then incubated at 65° C. for 1 hour with cyclodextrins that had been dissolved in PBS. As a control, PBS-loaded liposomes were generated by rehydration of the lipid film with PBS instead of citrate. The incubation at relatively high temperature (65° C.) enhanced the fluidity of the lipid bilayer, thus allowing the cyclodextrins to cross it. The suspensions were then dialyzed overnight in PBS to remove cyclodextrins that had not been incorporated into liposomes and analyzed for dansyl fluorescence. These experiments showed that >90% of each of these cyclodextrins were entrapped in the liposomes (see, for example, FIGS. 3A and 4). In the absence of a pH gradient, there was little incorporation of the same compounds into the liposomes (FIG. 3A). Light scattering showed that the preformed “empty” liposomes had a mean diameter of 98 nm with a narrow polydispersity index (<0.10) (FIG. 3B). Incorporation of the cyclodextrins just slightly increased the mean diameter to 105 nm without changing the polydispersity index (FIG. 3C). Cryo-transmission electron microscopy revealed that the structure of the liposomes following incorporation of cyclodextrins was unchanged except for an increased density within the liposomes, presumably reflecting the high concentration of cyclodextrins within them (FIG. 3C). In contrast, cyclodextrins incubated with control, PBS-containing liposomes with no transmembrane pH gradient, resulted in irregularly shaped, large vesicles, with no evidence of cyclodextrin incorporation within them (FIG. 3D). The change in shape observed with the control liposomes was presumably due to association of cyclodextrins with the lipid bilayer, leading to destabilization and “bloating” of the liposomal structure.

### Example 3: Small Hydrophobic Compounds Encapsulated within Chemically-Modified β-Cyclodextrins and Ferried Into Liposomes

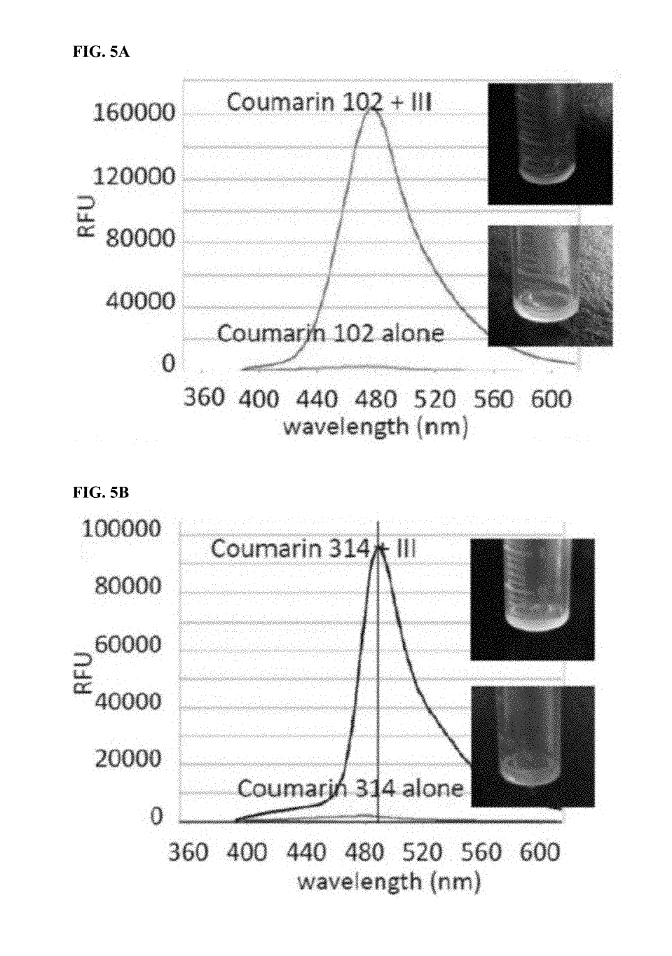

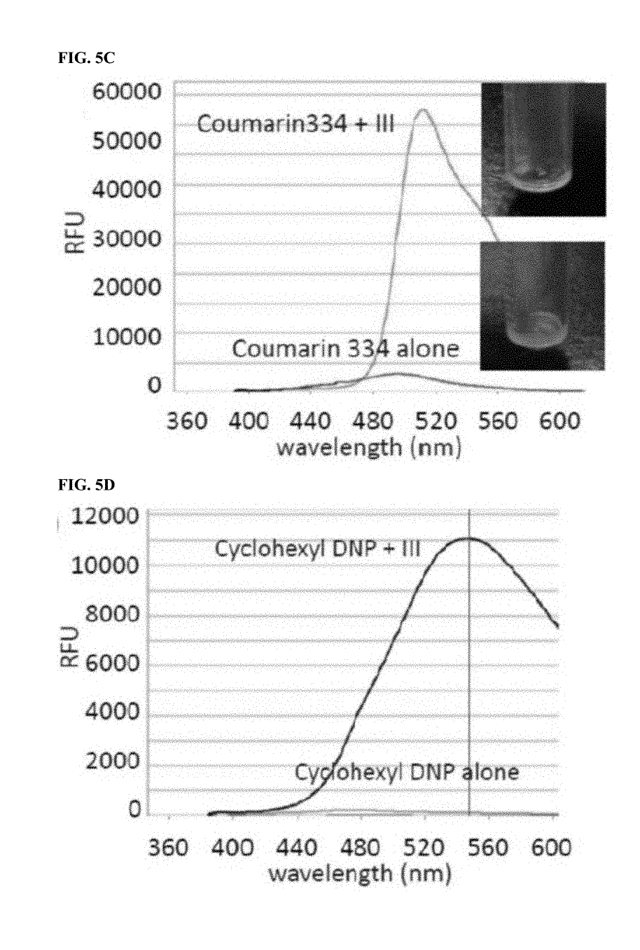

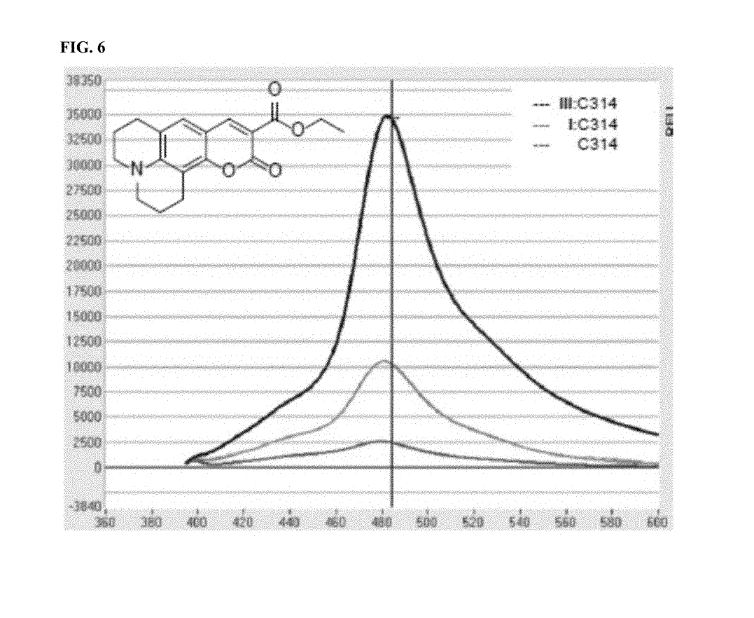

Organic dyes (coumarins) were used to determine whether the modified cyclodextrins could encapsulate and transport hydrophobic compounds across the liposome bilayer. Coumarins are very hydrophobic and a dramatic improvement in aqueous solubility was observed after they were encapsulated into 6′-mono-ethylenediamino-6′-deoxy-cyclodextrin (compound III). It was determined that the most efficient and convenient way to encapsulate the coumarins was by freeze drying (Cao et al. (2005) Drug Dev. Industr. Pharm. 31:747-756; Badr-Eldin et al. (2008) Eur. J. Pharm. Biopharm. 70:819-827). The solubility of the coumarins increased at least 10- to 20-fold (from 100 μg/mL to 1-2 mg/mL) through this procedure. Once dissolved, the cyclodextrin-coumarin complexes were incubated with pre-formed liposomes exactly as described above, using a pH gradient to drive the compounds across the bilayer. Following overnight dialysis to remove unincorporated complexes, the liposomes were subsequently disrupted with methanol and the coumarin fluorescence measured. As shown by fluorescence spectroscopy, all cyclodextrin-dye complexes were incorporated into liposomes with high efficiency (>95%; FIG. 5). To ascertain that this highly efficient loading was indeed due to active transportation of the complex across the lipid membrane and not due to enhanced water solubility only, the loading efficiencies of coumarin 314 in the absence of cyclodextrins was evaluated and it was found to be poorly incorporated into liposomes under the identical conditions (FIG. 6). Importantly, coumarin 314 encapsulated in unionizable native β-cyclodextrin only marginally improved the loading efficiency, despite substantially increased aqueous solubility of the dye. The incorporation of coumarin dyes into liposomes was easily discerned by the naked eye, as the coumarin-cyclodextrin liposomes were bright yellow, whereas control liposomes (made without cyclodextrins, for example) were colorless (FIGS. 5A-5C).

### Example 4: Chemotherapeutic Agents Encapsulated within Chemically-Modified 3-Cyclodextrins and Ferried into Liposomes

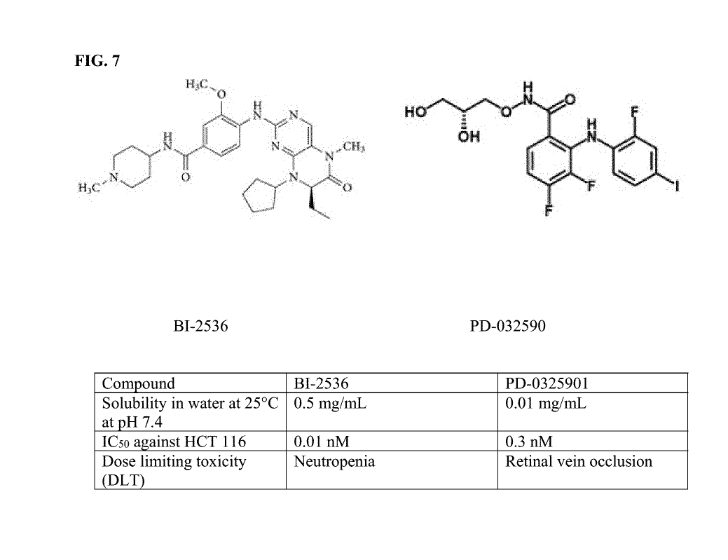

The ability of the amino-functionalized cyclodextrins to engender a liposomal formulation of BI-2536 was determined (FIG. 7). BI-2536, developed by Boehringer Ingelheim, is a highly selective inhibitor of polo-like kinase (PLK1), an enzyme required for the proper execution of mitosis (Steegmaier et al. (2007) Curr. Biol. 17:316-322; Lenart et al. (2007) Curr. Biol. 17:304-315; and Stewart et al. (2011) Exp. Hematol. 39:330-338). It has been shown that BI-2536 has potent tumoricidal activity against cancer cells, particularly those harboring mutations in TP53 (Sur et al. (2009) Proc. Natl. Acad. Sci. U.S.A. 106:3964-3969; Sanhaji et al. (2013) Cell Cycle 12:1340-1351; Meng et al. (2013) Gynecol. Oncol. 128:461-469; and Nappi et al. (2009) Canc. Res. 69:1916-1923). BI-2536 was the subject of several clinical trials in patients with cancers of the lung, breast, ovaries, and uterus (Mross et al. (2012) Br. J. Canc. 107:280-286; Ellis et al. (2013) Clin. Lung Canc. 14:19-27; Hofheinz et al. (2010) Clin. Canc. Res. 16:4666-4674; Sebastian et al. (2010) J. Thorac. Oncol. 5:1060-1067; Schoffski et al. (2010) Eur. J. Canc. 46:2206-2215; and Mross et al. (2008) J. Clin. Oncol. 26:5511-5517). Although it showed evidence of efficacy in cancer patients, its development was abandoned after Phase II trials revealed unacceptable toxicity (grade 4 neutropenia) at sub-therapeutic doses.

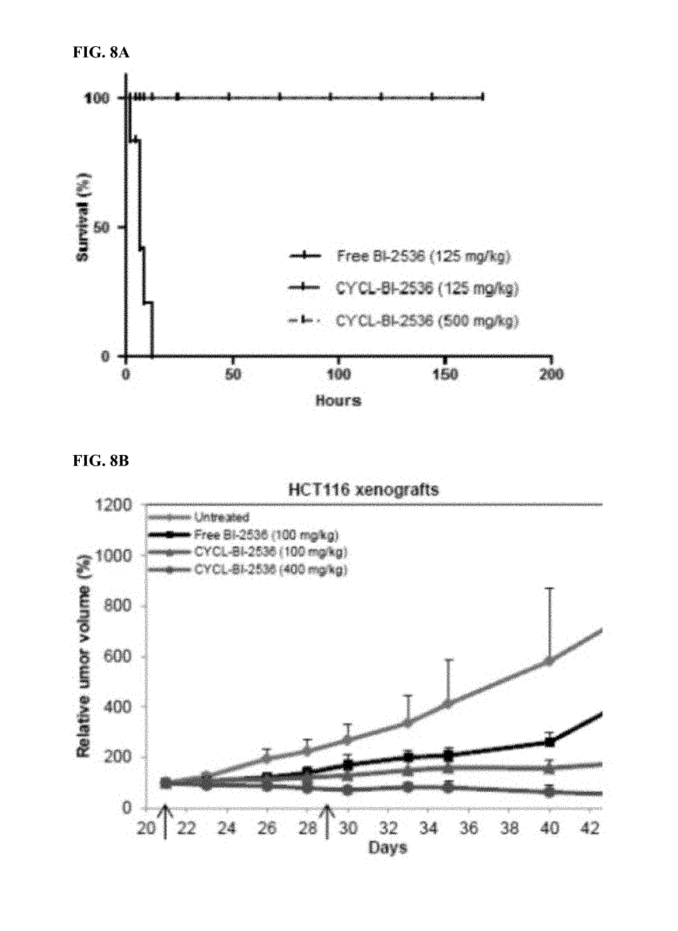

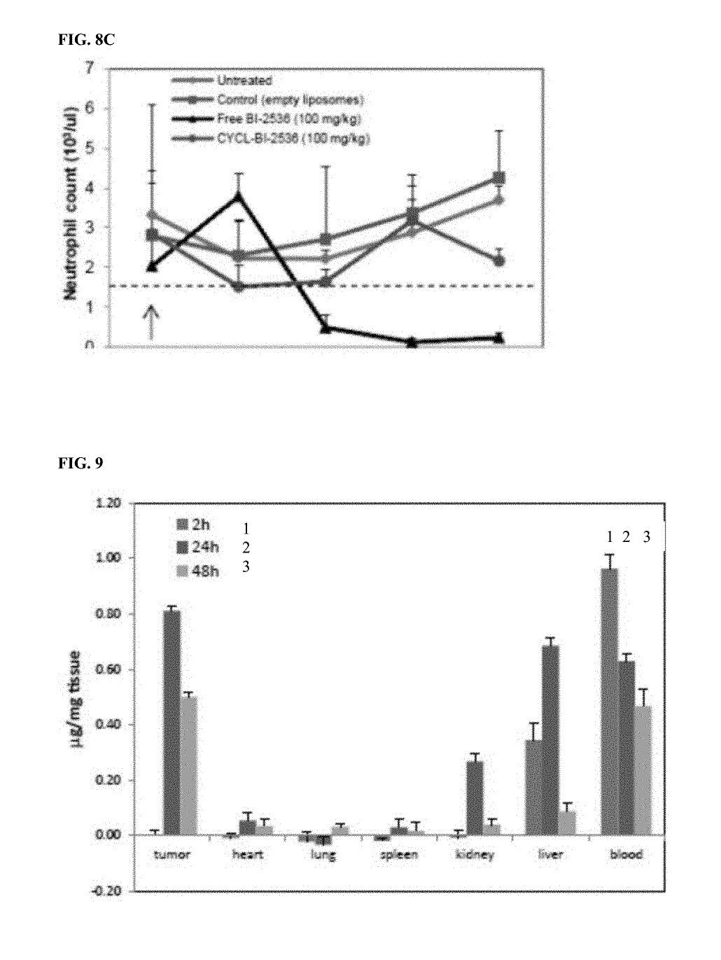

It was determined herein that aminated cyclodextrins V and VI dramatically improved the aqueous solubility of BI-2536. As with the coumarins, the BI-2536 complexes were determined to be reproducibly loadable into liposomes using compound VI, achieving stable aqueous solutions containing 10 mg/mL of drug. By comparison, the maximum aqueous solubility of free BI-2536 was determined to be 0.5 mg/mL. To assess the activity of cyclodextrin-encapsulated, liposomal (CYCL) forms of BI-2536, their effects were assessed in nude mice bearing subcutaneous xenografts of human HCT116 colorectal cancer cells. Three weeks after HCT116 cells were subcutaneously injected into the mice, they were treated with empty liposomes, free BI-2536, or CYCL-BI-2536. At the initiation of treatment, the tumors were already relatively large, averaging ˜300 mm³ and more closely mimicking clinical situations than small tumors. Severe acute toxicity was evident when the free drug was administered intravenously (iv) at 125 mg/kg: the mice became lethargic within minutes, their eyes turned glassy, they exhibited ruffled fur, and died a few hours later (FIG. 8A). Mice treated with a slightly lower dose of free BI-2536 (100 mg/kg) were somewhat lethargic immediately after drug administration, but survived. However, delayed toxicity, manifested as a drastic decrease in peripheral WBCs, was evident within 24-36 hours after free drug administration. This type of toxicity was identical to that observed in human clinical trials (Mross et al. (2008) J. Clin. Oncol. 26:5511-5517; Frost et al. (2012) Current Oncol. 19:e28-35; and Vose et al. (2013) Leuk. Lymphom. 54:708-713). Although toxic to the bone marrow, the free BI-2536 was able induce a significant anti-tumor response, slowing tumor growth by ˜30% after two doses at its maximum tolerated dose (MTD) (FIG. 7B). This efficacy was previously observed in other murine models (Steegmaier et al. (2007) Curr. Biol. 17:316-322; Nappi et al. (2009) Canc. Res. 69:1916-1923; Ackermann et al. (2011) Clin. Canc. Res. 17:731-741; Grinshtein et al. (2011) Canc. Res. 71:1385-1395; Liu et al. (2011) Anti-Canc. Drugs 22:444-453; and Ding et al. (2011) Canc. Res. 71:5225-5234) and provided the rationale for the clinical trials.

CYCL-BI-2536 proved far superior to the free form, both with respect to toxicity and efficacy. CYCL-BI-2536, even at a dose of 500 mg/kg, did not cause any noticeable adverse reactions; this dose was 4-fold higher than the dose of free drug, which killed every animal (FIG. 8A). At a dose of 100 mg/kg (equivalent to the MTD of the free drug), CYCL-BI-2536 induced a significantly improved tumor response, slowing tumor growth by nearly 80% after only two doses (FIG. 8B). At a dose of 400 mg/kg, the CYCL-BI-2536 resulted not only in slower growth, but also in partial regressions of tumors (FIG. 8B). The equivalent dose of the free CYCL-BI-2536 could not be administered because the mice could not survive a dose even close to this amount (FIG. 8B). Moreover, relatively little bone marrow toxicity resulted from treatment with CYCL-BI-2536 as the WBC decrease was much less and did not pose a risk to the animals (FIG. 8C). Finally, it was determined that CYCL-BI-2536 had much greater efficacy than the free drug against a second human colorectal cancer model, HCT116 cells with genetically inactivated TP53 alleles. In both cases, significant regressions were observed with the CYCL-form of the drug, but not with free drug.

To establish biodistribution and pharmacokinetics of the CYCL liposomes, liposomes loaded with coumarin 334 encapsulated in cyclodextrin VI were used to treat HCT116-bearing mice by intravenous (i.v.) injection. Samples from major tissues harvested at 2, 24, and 48 hours post-treatment were analyzed for their fluorescence. As expected, coumarin 334 was cleared from most of the tissues examined at 48 hours after treatment. Importantly, the agent encapsulated in liposomes persisted in the blood and tumor, which is consistent with the typical pharmacokinetics of PEGylated liposomes (FIG. 9).

The cyclodextrin-based loading method was also compared with the most common approaches to entrapping hydrophobic and insoluble agents in liposomes. First, direct entrapment of BI-2536 in the lipid bilayer was attempted. BI-2536 was co-evaporated with hydrogenated egg phosphatidylcholine-cholesterol-1,2-distearoyl-sn-glycero-3-phosphoethanolamine-N-[methoxy(polyethylene glycol)-2000 (DSPE-PEG 2000) to prepare a thin film, which was subsequently hydrated with 1 mL PBS and extruded through a 100-nm polycarbonate membrane at 700 psi to prepare small, unilamellar vesicles (average particle size (Z_{avg}) 126 nm; PDI 0.09). Upon overnight dialysis against PBS to remove unentrapped drug, the drug-containing liposomes rapidly swelled (Z_{avg} 539 nm; PDI 0.49) and released nearly 90% of the entrapped drug. Second, the lipid film was hydrated with an aqueous formulation of BI-2536 (passive loading). Hydration of the lipid film, followed by extrusion and dialyses, led to stable liposomes. However, the encapsulation efficiency was <10%, 20-fold less than achievable with the modified cyclodextrins described herein.

In order to further document the generality of this approach, PD-0325901, a mitogen-activated protein kinase kinase 1 (MEK1) inhibitor developed by Pfizer that was abandoned because it caused retinal vein occlusion (RVO) in Phase 2 trials, was evaluated (Brown et al. (2007) Canc. Chemo. Pharmacol. 59:671-679; LoRusso et al. (2010) Clin. Canc. Res. 16:1924-1937; Haura et al. (2010) Clin. Canc. Res. 16:2450-2457; and Huang et al. (2009) J. Ocul. Pharmacol. Therap. 25:519-530). Aminated cyclodextrins (FIG. 2, compounds VI and VII), as well as succinylated cyclodextrins (FIG. 2, compounds VIII and IX), were tested for their ability to encapsulate PD-0325901 and load them into acidic or basic liposomes, respectively. The free drug was exceedingly insoluble (0.1 mg/mL in water), and its solubility increased by nearly 40-fold after encapsulation into cyclodextrins. The best liposome loading was achieved with succinylated cyclodextrin IX and this formulation was tested in animals bearing human tumor xenografts, as described above for BI-2536. As with the BI-2536, PD-0325901 complexes were reproducibly loadable into liposomes, achieving stable solutions containing 5 mg/mL of drug.

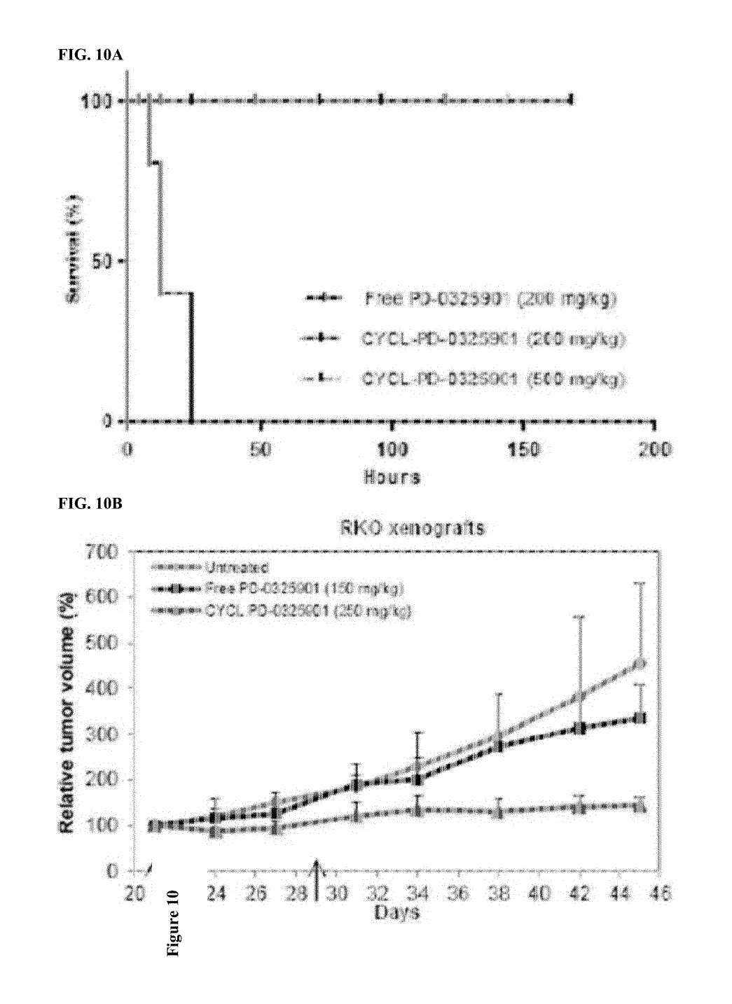

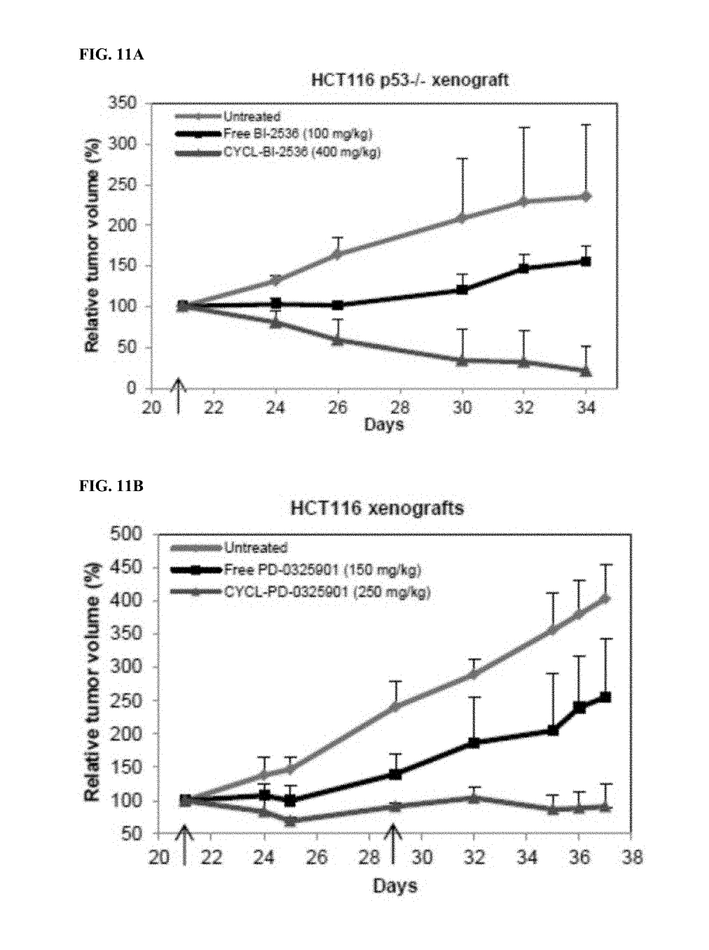

In order to assess the activity of cyclodextrin-complexed, liposomal (CYCL) forms of PD-0325901, their effects were analyzed in the RKO xenograft model. With the free drug, severe acute toxicity was evident. In previous studies, the free drug was administered by oral gavage, because its solubility is not sufficient for intravenous dosing. After oral gavage at 200 mg/kg, the treated animals were lethargic within minutes and over the next few hours they appeared hunched and always died within 24 hours (FIG. 10A). Mice treated with a slightly lower dose of free PD-0325901 (150 mg/kg) exhibited similar symptoms immediately after dosing but recovered over 24 hours. However, a single dose of free PD-0325901 was unable induce a dramatic anti-tumor response, slowing tumor growth only by ˜5% (FIG. 10B). This result was consistent with the those previously reported in other murine models; higher efficacy of the free drug required multiple doses.

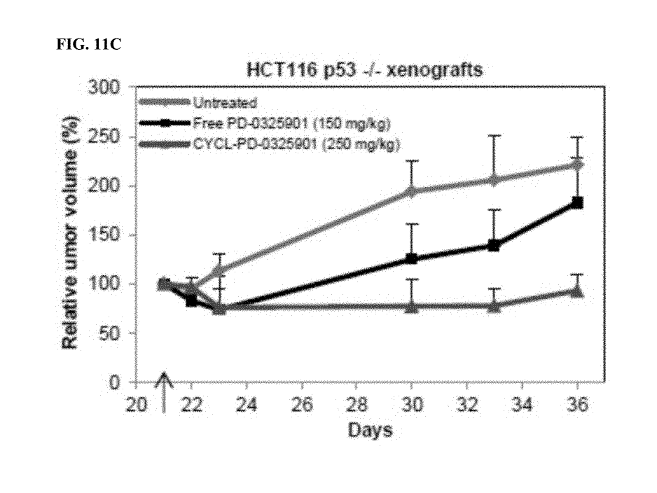

By contrast, CYCL-PD-0325901 proved far superior to the free drug. Even at a dose of 500 mg/kg, CYCL-PD-0325901 did not cause any noticeable adverse reactions; this dose was 2.5-fold higher than the dose of free drug which killed every animal (FIG. 10A). At a single dose of 250 mg/kg, the CYCL-PD-0325901 resulted not only in slower growth but also in partial regressions of tumors (FIG. 10B). Finally, CYCL-PD-0325901 was evaluated against two other human colorectal cancer models (HCT116 and its isogenic counterpart with genetically inactivated TP53 alleles) and higher efficacy and lower toxicity compared with free drug were similarly observed (FIG. 11).

The results described above demonstrate a general strategy for loading hydrophobic drugs into liposomes based on employing modified cyclodextrins with ionizable groups on their external surfaces (FIG. 2). The “pockets” of these cyclodextrins can encapsulate hydrophobic drugs and ferry them across the bilayer membrane of conventional liposomes using simple pH gradients. It has been demonstrated herein that many types of compounds can successfully be encapsulated into the modified cyclodextrins, including coumarin dyes and drugs of potential clinical importance (FIGS. 3, 5, 8, and 10). This incorporation not only dramatically increased the aqueous solubility of all these compounds but also allowed them to be remotely loaded into liposomes. Moreover, the loaded liposomes exhibited substantially less toxicity (FIG. 8) and greater activity (FIGS. 8 and 10) when tested in mouse models of cancer.

Previous attempts to combine cyclodextrin inclusion complexes with liposomes were limited to passive loading of insoluble drugs (Zhu et al. (2013) J. Pharm. Pharmacol. 65(8):1107-1117; Malaekeh-Nikouei and Davies (2009) PDA J. Pharm. Sci. Technol. 63:139-148; Rahman et al. (2012) Drug Deliv. 19:346-353; Ascenso et al. (2013) J. Liposome Res. 23:211-219; Dhule et al. (2012) Nanomedicine 8:440-451; Lapenda et al. (2013) J. Biomed. Nanotechnol. 9:499-510; and Mendonca et al. (2012) AAPS PharmSciTech. 13:1355-1366) or active loading of soluble drugs (Modi et al. (2012) J. Control Release 162:330-339). Passive loading often leads to undesirable membrane incorporation, lowering liposome stability, and is much less efficient than active loading. For example, the drug to lipid ratios achieved through the approaches described herein ranged from 0.4 to 0.6, which is more than 1,000-fold higher than the drug to lipid ratios commonly achieved through passive loading (Zhu et al. (2013) J. Pharm. Pharmacol. 65(8): 1107-1117; Malaekeh-Nikouei and Davies (2009) PDA J. Pharm. Sci. Technol. 63:139-148; Rahman et al. (2012) Drug Deliv. 19:346-353; Ascenso et al. (2013) J. Liposome Res. 23:211-219; Dhule et al. (2012) Nanomedicine 8:440-451; and Modi et al. (2012) J. Control Release 162:330-339).

Because many of the most promising drugs developed today and in the past are relatively insoluble, the approaches described herein can be broadly applicable. The approaches not only increase water solubility, but also enhances the selectivity of drug delivery to tumors through an enhanced permeability and retention (EPR) effect (Wang et al. (2012) Annu. Rev. Med. 63:185-198; Peer et al. (2007) Nat. Nanotech. 2:751-760; Gubernator (2011) Exp. Opin. Drug Deliv. 8:565-580; Huwyler et al. (2008) Int. J. Nanomed. 3:21-29; Maruyama et al. (2011) Adv. Drug Deliv. Rev. 63:161-169; Musacchio and Torchilin (2011) Front. Biosci. 16:1388-1412; Baryshnikov (2012) Vest. Ross. Akad. Med. Nauk. 23-31; and Torchilin (2005) Nat. Rev. Drug Disc. 4:145-160). The results using BI-2536 provide a striking example of the benefits of these attributes-simultaneously increasing solubility and selective tumor delivery that result in much less toxicity and increased efficacy. These strategies therefore have the capacity to “rescue” drugs that fail at one of the last steps in the laborious and expensive process of drug development, allowing administration at higher doses and with less toxicity than otherwise obtainable.

### INCORPORATION BY REFERENCE

The contents of all references, patent applications, patents, and published patent applications, as well as the Figures and the Sequence Listing, cited throughout this application are hereby incorporated by reference. It will be understood that, although a number of patent applications, patents, and other references are referred to herein, such reference does not constitute an admission that any of these documents forms part of the common general knowledge in the art.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A liposome composition comprising a cyclodextrin, a therapeutic agent, and a liposome, wherein the liposome encapsulates a cyclodextrin having at least one hydroxyl chemical group facing the liposome internal phase replaced with an ionizable chemical group and wherein the cyclodextrin encapsulates the therapeutic agent.

2. The liposome composition of claim 1, wherein at least one α-D-glucopyranoside unit of the cyclodextrin has at least one hydroxyl chemical group selected from the group consisting of C2, C3, and C6 hydroxyl chemical groups that are replaced with an ionizable chemical group.

3. The liposome composition of claim 2, wherein at least one α-D-glucopyranoside unit of the cyclodextrin has at least two hydroxyl chemical groups selected from the group consisting of C2, C3, and C6 hydroxyl chemical groups that are replaced with ionizable chemical groups, wherein the C2, C3, and C6 hydroxyl chemical groups of at least one α-D-glucopyranoside unit of the cyclodextrin that are replaced with ionizable chemical groups.

4. (canceled)

5. The liposome composition of claim 2, wherein the at least one α-D-glucopyranoside unit of the cyclodextrin is selected from the group consisting of two, three, four, five, six, seven, eight, and all α-D-glucopyranoside units of the cyclodextrin.

6. The liposome composition of claim 1, wherein the ionizable chemical group is the same at all replaced positions.

7. The liposome composition of claim 1, wherein the ionizable chemical group is a weakly basic functional group or a weakly acidic functional group.

8. The liposome composition of claim 7, wherein the weakly basic functional group (X) has a pKₐ between 6.5 and 8.5 according to CH3-X.

9. The liposome composition of claim 7, wherein the weakly acidic functional groups (Y) have a pKₐ between 4.0 and 6.5 according to CH₃—Y.

10. The liposome composition of claim 7, wherein the weakly basic or weakly acidic functional groups are selected from the group consisting of amino, ethylene diamino, dimethyl ethylene diamino, dimethyl anilino, dimethyl naphthylamino, succinyl, carboxyl, sulfonyl, and sulphate functional groups.

11. The liposome composition of claim 1, wherein the cyclodextrin has a pKₐ₁ of between 4.0 and 8.5.

12. The liposome composition of claim 1, wherein the composition is a liquid or solid pharmaceutical formulation.

13. The liposome composition of claim 1, wherein the therapeutic agent is neutrally charged or hydrophobic.

14. The liposome composition of claim 1, wherein the therapeutic agent is a chemotherapeutic agent or a small molecule.

15. (canceled)

16. The liposome composition of claim 1, wherein the cyclodextrin is selected from the group consisting of β-cyclodextrin, α-cyclodextrin, and γ-cyclodextrin.

17. (canceled)

18. A kit comprising a liposome composition of claim 1, and instructions for use.

19. A method of treating a subject having a cancer comprising administering to the subject a therapeutically effective amount of a liposome composition of claim 1.

20. The method of claim 19, wherein the therapeutic agent is a chemotherapeutic agent.

21. The method of claim 19, wherein the liposome composition is administered by injection subcutaneously or intravenously.

22. The method of claim 19, wherein the subject is a mammal, wherein the mammal is a human.

23. (canceled)
